(19) United States
(12) Patent Application Publication
(10) Pub. No.: US 2008/0249105 A1
(43) Pub. Date: Oct. 9, 2008

(54) Pyrrolopyridine-Substituted Benzol Derivatives for Treating Cardiovascular Diseases

(75) Inventors: Samir Bennabi, Lyon (FR); Heike Heckroth, Odenthal (DE); Hartmut Schirok, Wuppertal (DE); Joachim Mittendorf, Wuppertal (DE); Raimund Kast, Wuppertal (DE); Johannes-Peter Stasch, Solingen (DE); Mark Jean Gnoth, Mettmann (DE); Klaus Minter, Wulfrath (DE); Dieter Lang, Velbert (DE); Santiago Figueroa Perez, Leverkusen (DE); Marcus Bauser, Wuppertal (DE); Achim Feurer, Liestal (CH); Heimo Ehmke, Hamburg (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer Healthcare AG, Leverkusen (DE)

(21) Appl. No.: 10/582,184

(22) PCT Filed: Nov. 26, 2004
(86) PCT No.: PCT/EP04/13430

§ 371 Date: Sep. 26, 2007

(30) Foreign Application Priority Data
Dec. 9, 2003 (DE) 103 57 510.3

(51) Int. Cl. A61K 31/496; C07D 471/02; A61K 31/437; A61P 15/10; A61P 9/00; C07D 401/14

(52) U.S. Cl. 514/253.04; 546/113; 514/300; 544/362

(57) ABSTRACT

The invention relates to heteroaryl-substituted benzenes, to a process for their preparation and to their use for preparing medicaments for the treatment and/or prophylaxis of diseases in humans and animals, in particular cardiovascular disorders.

The invention relates to heteroaryl-substituted benzenes, to a process for their preparation and to their use for preparing medicaments for the treatment and/or prophylaxis of diseases in humans and animals, in particular cardiovascular disorders.

An increase in the intracellular calcium concentration is one of the main factors triggering the contraction of the vascular musculature (Somlyo, A. P. and Himpens, B. FASEB J. 1989, 3, 2266-2276). This is effected primarily by agonists, such as, for example, phenylephrine or thromboxane A2 which, after stimulation of the phosphatidylinositol cascade, cause the release of calcium from the sarcoplasmatic reticulum. The elevated intracellular calcium activates the MLC kinase (myosin light-chain kinase) which phosphorylates the MLC subunits of the myosin molecule (Kamm, K. H. and Stull, J. T., Annu. Rev. Pharmacol. Toxicol. 1985, 25, 593-603). MLC phosphorylation induces the contraction of smooth muscles, MLC dephosphorylation after reduction of the intracellular calcium concentration results in the relaxation of the vessel.

In addition to the calcium-dependent MLC phosphorylation, there is a further, central but calcium-independent, regulation mechanism of the vascular tone. This is the Rho/Rho kinase signal path (Noda, M. et al., FEBS Lett. 1995, 367, 246-250; Uehata, M. et al., Nature 1997, 389, 990-994; Fukata, Y. et al., Trends in Pharmacological Sciences 2001, 22, 32-39). The binding of agonists such as, for example, phenylephrine or thromboxane A2 to their receptors results in the activation of the small G-proteins Rho which then interact with and activate Rho kinase. The activated Rho kinase inhibits myosin phosphatase following phosphorylation of a subunit of the enzyme. At the same time, Rho kinase phosphorylates MLC at the position which is also phosphorylated by MLC kinase. Inhibition of myosin phosphatase and phosphorylation of MLC induces the vascular musculature to contract. In contrast, inhibition of Rho kinase leads to a relaxation of the vessels. Accordingly, inhibitors of Rho kinase lower the blood pressure and increase coronary perfusion.

In addition, inhibitors of Rho kinase cause inhibition of growth of tumour cells and metastases (Itoh et al. Nat. Med. 1999, 5, 221; Somlyo et al. Biochem. Biophys. Res. Commun. 2000, 269, 652) and inhibit angiogenesis (Uchida et al. Biochem. Biophys. Res. Commun. 2000, 269, 633; Gingras et al. Biochem. J. 2000, 348 Vol. 2, 273).

Structures similar to the compounds according to the invention are only known from other indications. Thus, for example, US 2001/0020030 A1 discloses substituted thienopyridines and thienopyrimidines for treating inflammatory disorders, WO 02/32872 discloses nitrogenous aromatic cyclic compounds as inhibitors of neovascularization.

The present invention provides compounds of the formula

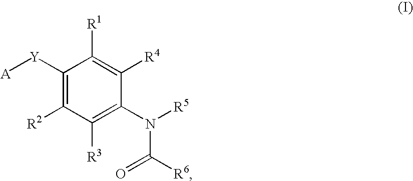

in which
A represents a radical

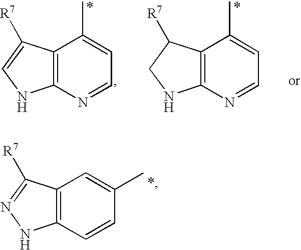

in which,
R⁷ represents hydrogen, halogen, cyano, (C₁-C₆)-alkyl, (C₃-C₆)-cycloalkyl, phenyl or 5- or 6-membered heteroaryl,
   where alkyl, cycloalkyl, phenyl or 5- or 6-membered heteroaryl may be substituted by amino, hydroxyl, halogen, (C₁-C₃)-alkyl, (C₁-C₃)-alkoxy or (C₁-C₆)-alkylamino,
and
* represents the point of attachment to Y,
Y represents O or NH,
R¹ and R² independently of one another represent hydrogen, halogen, cyano or (C₁-C₃)-alkyl,
R³ and R⁴ independently of one another represent hydrogen, fluorine, chlorine or methyl,
R⁵ represents hydrogen or (C₁-C₆)-alkyl,
R⁶ represents a radical selected from the group consisting of:
   (C₁-C₆)-alkyl which is substituted by amino, hydroxyl, (C₁-C₆)-alkoxy, (C₁-C₆)-alkylthio, (C₁-C₆)-alkylamino, (C₃-C₈)-cycloalkylamino, (C₁-C₆)-alkylcarbonylamino, (C₁-C₆)-alkoxycarbonyl, (C₃-C₈)-cycloalkyl, (C₆-C₁₀)-aryl, 5- to 10-membered heteroaryl or 5- to 10-membered heterocyclyl,
      where alkylamino, cycloalkylamino or aryl for their part may be substituted by amino, hydroxyl, halogen, (C₁-C₆)-alkoxy, (C₁-C₆)-alkylamino or (C₆-C₁₀)-aryl,
   (C₁-C₆)-alkoxy which may be substituted by amino, hydroxyl or (C₁-C₆)-alkylamino, dimethylaminoethylamino,
   (C₃-C₈)-cycloalkyl, 5- to 10-membered heterocyclyl or 5- to 10-membered heterocyclyloxy,
      where cycloalkyl, heterocyclyl or heterocyclyloxy may be substituted by amino, hydroxyl, (C₁-C₆)-alkyl, (C₁-C₆)-alkylamino, oxo or benzyloxy,
   and (C₆-C₁₀)-aryl or 5- to 10-membered heteroaryl,
      where aryl or heteroaryl may be substituted by amino, hydroxyl, halogen, cyano, (C₁-C₆)-alkyl, which for its part may be substituted by amino or (C₁-C₆)-alkylamino, (C₁-C₆)-alkoxy, (C₁-C₆)-alkylamino or (C₁-C₆)-alkoxycarbonyl,
         and their salts, hydrates, hydrates of the salts and solvates.

Compounds according to the invention are the compounds of the formula (I) and their salts, solvates and solvates of the salts; the compounds of the formulae given below embraced by formula (I) and their salts, solvates and solvates of the salts and the compounds given below as embodiments and embraced by formula (I) and their salts, solvates and solvates of the salts, if the compounds given below and embraced by formula (I) are not already salts, solvates and solvates of the salts.

Depending on their structure, the compounds according to the invention can exist in stereoisomeric forms (enantiomers, diastereomers). Accordingly, the invention relates to the enantiomers or diastereomers and to their respective mixtures. The stereoisomerically uniform components can be isolated in a known manner from such mixtures of enantiomers and/or diastereomers.

Depending on the structure of the compounds, the invention also relates to tautomers of the compounds.

In the context of the invention, preferred salts are physiologically acceptable salts of the compounds according to the invention.

Physiologically acceptable salts of the compounds (I) include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid or benzenesulphonic acid, naphthalenedisulphonic acid, acetic acid, propionic acid, lactic acid, tartaric acid, malic acid, citric acid, fumaric acid, maleic acid, trifluoroacetic acid and benzoic acid.

Physiologically acceptable salts of the compounds (I) also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, dihydroabiethylamine, arginine, lysine, ethylenediamine and methylpiperidine.

In the context of the invention, solvates are those forms of the compounds which, in solid or liquid state, form a complex by coordination with solvent molecules. Hydrates are a specific form of solvates where the coordination is with water.

In the context of the present invention, the substituents are as defined below, unless specified otherwise:

alkyl per se and “alk” and “alkyl” in alkoxy, alkylthio, alkylamino, alkylcarbonylamino and alkoxycarbonyl represent a straight-chain or branched alkyl radical having generally 1 to 6, preferably 1 to 4, particularly preferably 1 to 3, carbon atoms, by way of example and by way of preference methyl, ethyl, n-propyl, isopropyl, tert-butyl, n-pentyl and n-hexyl.

By way of example and by way of preference, alkoxy represents methoxy, ethoxy, n-propoxy, isopropoxy, tert-butoxy, n-pentoxy and n-hexoxy.

By way of example and by way of preference, alkylthio represents methylthio, ethylthio, n-propylthio, isopropylthio, tert-butylthio, n-pentylthio and n-hexylthio.

Alkylamino represents an alkylamino radical having one or two alkyl substituents (selected independently of one another). (C₁-C₃)-alkylamino represents, for example, a monoalkylamino radical having 1 to 3 carbon atoms or a dialkylamino radical having in each case 1 to 3 carbon atoms per alkyl substituent. By way of example and by way of preference methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino may be mentioned.

By way of example and by way of preference, alkylcarbonylamino represents methylcarbonylamino, ethylcarbonylamino, n-propylcarbonylamino, isopropylcarbonylamino, tert-butylcarbonylamino, n-pentylcarbonylamino and n-hexylcarbonylamino.

By way of example and by way of preference, alkoxycarbonyl represents methoxycarbonyl, ethoxycarbonyl, n-propoxycarbonyl, isopropoxycarbonyl, tert-butoxycarbonyl, n-pentoxycarbonyl and n-hexoxycarbonyl.

Cycloalkyl represents a cycloalkyl group having generally 3 to 8, preferably 5 to 7, carbon atoms, by way of example and by way of preference cyclopropyl, cyclobutyl, cyclopentyl, cyclohexyl and cycloheptyl.

Cycloalkylamino represents a cycloalkylamino group having generally 3 to 8, preferably 5 to 7, carbon atoms, by way of example and by way of preference cyclopropylamino, cyclobutylamino, cyclopentylamino, cyclohexylamino and cycloheptylamino.

Aryl represents a mono- to tricyclic aromatic carbocyclic radical having generally 6 to 14 carbon atoms, preferably 6 or 10 carbon atoms; by way of example and by way of preference phenyl, naphthyl and phenanthrenyl.

Heteroaryl represents an aromatic mono- or bicyclic radical having generally 5 to 10, preferably 5 to 6, ring atoms and up to 5, preferably up to 4, heteroatoms from the group consisting of S, O and N, by way of example and by way of preference thienyl, furyl, pyrrolyl, pyrazolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl.

Heterocyclyl per se and in heterocyclyloxy represents a mono- or polycyclic, preferably mono- or bicyclic, non-aromatic heterocyclic radical having 5 to 10, generally 5 to 8, preferably 5 or 6, ring atoms and up to 3, preferably up to 2, heteroatoms and/or hetero groups from the group consisting of N, O, S, SO, SO₂. The heterocyclyl radicals may be saturated or partially unsaturated. Preference is given to 5- or 6-membered monocyclic saturated heterocyclyl radicals having up to two heteroatoms from the group consisting of O, N and S, such as, by way of example and by way of preference, tetrahydrofuran-2-yl, tetrahydrothienyl, pyrrolidin-2-yl, pyrrolidin-3-yl, pyrrolinyl, pyranyl, piperidin-1-yl, piperidin-2-yl, piperidin-3-yl, piperidin-4-yl, thiopyranyl, morpholin-1-yl, morpholin-2-yl, morpholin-3-yl, perhydroazepinyl, piperazin-1-yl, piperazin-2-yl.

By way of example and by way of preference, heterocyclyloxy represents tetrahydrofuranyloxy, tetrahydrothienyloxy, pyrrolidinyloxy, pyrrolinyloxy, pyranyloxy, piperidinyloxy, thiopyranyloxy, morpholinyloxy, perhydroazepinyloxy, piperazinyloxy.

Halogen represents fluorine, chlorine, bromine and iodine.

If radicals in the compounds according to the invention are substituted, the radicals can be mono- or polysubstituted by identical or different substituents unless otherwise specified. A substition by up to three identical or different substituents is preferred. Very particular preference is given to substitution with one substituent.

Preference is given to compounds of the formula (I)

in which
A represents a radical

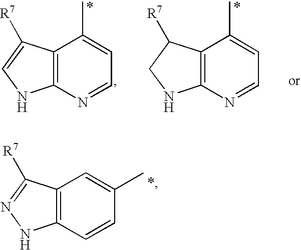

in which,
R⁷ represents hydrogen, halogen, cyano, (C₁-C₆)-alkyl, (C₃-C₆)-cycloalkyl, phenyl or 5- or 6-membered heteroaryl,
   where alkyl, cycloalkyl, phenyl or 5- or 6-membered heteroaryl may be substituted by amino, hydroxyl, halogen, (C₁-C₃)-alkyl, (C₁-C₃)-alkoxy or (C₁-C₆)-alkylamino,
and
* represents the point of attachment to Y,
Y represents O or NH,
R¹ and R² independently of one another represent hydrogen, halogen, cyano or (C₁-C₃)-alkyl,
R³ and R⁴ independently of one another represent hydrogen, fluorine, chlorine or methyl,
R⁵ represents hydrogen or (C₁-C₆)-alkyl,
R⁶ represents a radical selected from the group consisting of:
   (C₁-C₆)-alkyl which is substituted by amino, hydroxyl, (C₁-C₆)-alkoxy, (C₁-C₆)-alkylthio, (C₁-C₆)-alkylamino, (C₃-C₈)-cycloalkylamino, (C₁-C₆)-alkylcarbonylamino, (C₁-C₆)-alkoxycarbonyl, (C₃-C₈)-cycloalkyl, (C₆-C₁₀)-aryl, 5- to 10-membered heteroaryl or 5- to 10-membered heterocyclyl,
      where alkylamino, cycloalkylamino or aryl for their part may be substituted by amino, hydroxyl, halogen, (C₁-C₆)-alkoxy, (C₁-C₆)-alkylamino or (C₆-C₁₀)-aryl,
   (C₁-C₆)-alkoxy which may be substituted by amino, hydroxyl or (C₁-C₆)-alkylamino,
   (C₃-C₈)-cycloalkyl, 5- to 10-membered heterocyclyl or 5- to 10-membered heterocyclyloxy,
      where cycloalkyl, heterocyclyl or heterocyclyloxy may be substituted by amino, hydroxyl, (C₁-C₆)-alkyl, (C₁-C₆)-alkylamino, oxo or benzyloxy,
   and (C₆-C₁₀)-aryl or 5- to 10-membered heteroaryl,
      where aryl or heteroaryl may be substituted by amino, hydroxyl, halogen, cyano, (C₁-C₆)-alkyl, which for its part may be substituted by amino or (C₁-C₆)-alkylamino, (C₁-C₆)-alkoxy, (C₁-C₆)-alkylamino or (C₁-C₆)-alkoxycarbonyl,
         and their salts, hydrates, hydrates of the salts and solvates.

Preference is also given to compounds of the formula (I)

in which
A represents a radical

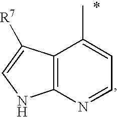

in which
R⁷ represents hydrogen, chlorine or methyl,
and
* represents the point of attachment to Y,
Y represents O,
R¹ and R² independently of one another represent hydrogen, fluorine or chlorine,
R³ and R⁴ independently of one another represent hydrogen or fluorine,
R⁵ represents hydrogen,
R⁶ represents a radical selected from the group consisting of:
   (C₁-C₆)-alkyl which is substituted by amino, hydroxyl, (C₁-C₆)-alkoxy, (C₁-C₆)-alkylthio, (C₁-C₆)-alkylamino, (C₅-C₆)-cycloalkylamino, (C₁-C₆)-alkylcarbonylamino, (C₁-C₆)-alkoxycarbonyl, phenyl, 5- or 6-membered heteroaryl or 5- or 6-membered heterocyclyl,
      where alkylamino, cycloalkylamino or phenyl for their part may be substituted by hydroxyl, halogen, (C₁-C₃)-alkoxy, (C₁-C₃)-alkylamino or phenyl,
   (C₁-C₆)-alkoxy which may be substituted by amino or (C₁-C₆)-alkylamino, cyclopentyl, cyclohexyl, 5- or 6-membered heterocyclyl or 5- or 6-membered heterocyclyloxy,
      where cyclopentyl, cyclohexyl, heterocyclyl or heterocyclyloxy may be substituted by amino, hydroxyl, (C₁-C₃)-alkyl, oxo or benzyloxy,
   and phenyl, thienyl, furyl, pyrrolyl, pyrazolyl, thiazolyl, oxazolyl, imidazolyl, pyridyl, pyrimidyl or pyridazinyl,
      where phenyl, thienyl, furyl, pyrrolyl, pyrazolyl, thiazolyl, oxazolyl, imidazolyl, pyridyl, pyrimidyl or pyridazinyl may be substituted by amino, hydroxyl, halogen, cyano, (C₁-C₃)-alkyl, which for its part may be substituted by amino or (C₁-C₆)-alkylamino, (C₁-C₃)-alkoxy or (C₁-C₃)-alkoxycarbonyl,
         and their salts, hydrates, hydrates of the salts and solvates.

Particular preference is given to compounds of the formula (I)

in which
A represents a radical

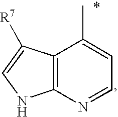

in which
R⁷ represents hydrogen, chlorine or methyl
and
* represents the point of attachment to Y,
Y represents O,
R¹ and R² independently of one another represent hydrogen or fluorine,
R³ and R⁴ represent hydrogen,
R⁵ represents hydrogen,
R⁶ represents a radical selected from the group consisting of:
   (C₁-C₆)-alkyl which is substituted by amino, hydroxyl, (C₁-C₆)-alkylamino, cyclohexylamino or piperidinyl,
      where alkylamino or cyclohexylamino for their part may be substituted by hydroxyl or phenyl,
   (C₁-C₆)-alkoxy which may be substituted by amino or (C₁-C₆)-alkylamino, cyclopentyl, piperazinyl, piperidinyl, pyrrolidinyl, piperidinyloxy or pyrrolidinyloxy,
      where cyclopentyl, piperazinyl, piperidinyl, pyrrolidinyl, piperidinyloxy or pyrrolidinyloxy may be substituted by amino, hydroxyl, (C₁-C₃)-alkyl or benzyloxy,
   and phenyl or thienyl,
      where phenyl or thienyl may be substituted by (C₁-C₃)-alkyl which for its part may be substituted by amino or (C₁-C₆)-alkylamino,
         and their salts, hydrates, hydrates of the salts and solvates.

The present invention also provides compounds of the formula (I)

in which
A represents a radical

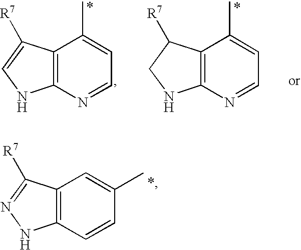

in which
R⁷ represents hydrogen, (C₁-C₆)-alkyl, (C₃-C₆)-cycloalkyl, phenyl or 5- or 6-membered heteroaryl,
   where alkyl, cycloalkyl, phenyl or 5- or 6-membered heteroaryl may be substituted by amino, hydroxyl, halogen, (C₁-C₃)-alkyl, (C₁-C₃)-alkoxy or (C₁-C₆)-alkylamino,
and
* represents the point of attachment to Y,
Y represents O or NH,
R¹ and R² independently of one another represent hydrogen, halogen, cyano or (C₁-C₃)-alkyl,
R³ and R⁴ independently of one another represent hydrogen, fluorine, chlorine or methyl,
R⁵ represents hydrogen or (C₁-C₆)-alkyl,
R⁶ represents a radical selected from the group consisting of:
   (C₁-C₆)-alkyl which is substituted by amino, hydroxyl, (C₁-C₆)-alkoxy, (C₁-C₆)-alkylthio, (C₁-C₆)-alkylamino, (C₃-C₈)-cycloalkylamino, (C₁-C₆)-alkylcarbonylamino, (C₁-C₆)-alkoxycarbonyl, (C₃-C₈)-cycloalkyl, (C₆-C₁₀)-aryl, 5- to 10-membered heteroaryl or 5- to 10-membered heterocyclyl,
      where alkylamino, cycloalkylamino or aryl for their part may be substituted by amino, hydroxyl, halogen, (C₁-C₆)-alkoxy, (C₁-C₆)-alkylamino or (C₆-C₁₀)-aryl,
   (C₁-C₆)-alkoxy which may be substituted by amino, hydroxyl or (C₁-C₆)-alkylamino, —NHR⁸,
      where
      R⁸ represents (C₁-C₆)-alkyl which may be substituted by amino, hydroxyl or (C₁-C₆)-alkylamino,
   (C₃-C₈)-cycloalkyl, 5- to 10-membered heterocyclyl or 5- to 10-membered heterocyclyloxy,
      where cycloalkyl, heterocyclyl or heterocyclyloxy may be substituted by amino, hydroxyl, (C₁-C₆)-alkyl, (C₁-C₆)-alkylamino, oxo or benzyloxy,
   and (C₆-C₁₀)-aryl or 5- to 10-membered heteroaryl,
      where aryl or heteroaryl may be substituted by amino, hydroxyl, halogen, cyano, (C₁-C₆)-alkyl, which for its part may be substituted by amino or (C₁-C₆)-alkylamino, (C₁-C₆)-alkoxy, (C₁-C₆)-alkylamino or (C₁-C₆)-alkoxycarbonyl,
         and their salts, hydrates, hydrates of the salts and solvates.

The present invention also provides compounds of the formula (I)

in which
A represents a radical

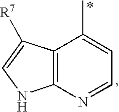

in which
R⁷ represents hydrogen or methyl,
and
* represents the point of attachment to Y,
Y represents O,
R¹ and R² independently of one another represent hydrogen, fluorine or chlorine,
R³ and R⁴ independently of one another represent hydrogen or fluorine,
R⁵ represents hydrogen,
R⁶ represents a radical selected from the group consisting of:
   (C₁-C₆)-alkyl which is substituted by amino, hydroxyl, (C₁-C₆)-alkoxy, (C₁-C₆)-alkylthio, (C₁-C₆)-alkylamino, (C₅-C₆)-cycloalkylamino, (C₁-C₆)-alkylcarbonylamino, (C₁-C₆)-alkoxycarbonyl, phenyl, 5- or 6-membered heteroaryl or 5- or 6-membered heterocyclyl,
      where alkylamino, cycloalkylamino or phenyl for their part may be substituted by hydroxyl, halogen, (C₁-C₃)-alkoxy, (C₁-C₃)-alkylamino or phenyl,
   (C₁-C₆)-alkoxy which may be substituted by amino or (C₁-C₆)-alkylamino, —NHR⁸,
      where R⁸ represents (C₁-C₆)-alkyl which may be substituted by amino or (C₁-C₆)-alkylamino,
   cyclopentyl, cyclohexyl, 5- or 6-membered heterocyclyl or 5- or 6-membered heterocyclyloxy,
      where cyclopentyl, cyclohexyl, heterocyclyl or heterocyclyloxy may be substituted by amino, hydroxyl, (C₁-C₃)-alkyl, oxo or benzyloxy,
   and phenyl, thienyl, furyl, pyrrolyl, pyrazolyl, thiazolyl, oxazolyl, imidazolyl, pyridyl, pyrimidyl or pyridazinyl,
      where phenyl, thienyl, furyl, pyrrolyl, pyrazolyl, thiazolyl, oxazolyl, imidazolyl, pyridyl, pyrimidyl or pyridazinyl may be substituted by amino, hydroxyl, halogen, cyano, (C₁-C₃)-alkyl, which for its part may be substituted by amino or (C₁-C₆)-alkylamino, (C₁-C₃)-alkoxy or (C₁-C₃)-alkoxycarbonyl,
         and their salts, hydrates, hydrates of the salts and solvates.

The present invention also provides compounds of the formula (I)

in which
A represents a radical

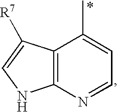

in which
R⁷ represents hydrogen or methyl,
and
* represents the point of attachment to Y,
Y represents O,
R¹ and R² independently of one another represent hydrogen or fluorine,
R³ and R⁴ represent hydrogen,
R⁵ represents hydrogen,
R⁶ represents a radical selected from the group consisting of:
   (C₁-C₆)-alkyl which is substituted by amino, hydroxyl, (C₁-C₆)-alkylamino, cyclohexylamino or piperidinyl,
      where alkylamino or cyclohexylamino for their part may be substituted by hydroxyl or phenyl,
   (C₁-C₆)-alkoxy which may be substituted by amino or (C₁-C₆)-alkylamino, —NHR⁸,
      where R⁸ represents (C₁-C₆)-alkyl which may be substituted by amino or (C₁-C₆)-alkylamino,
   cyclopentyl, piperazinyl, piperidinyl, pyrrolidinyl, piperidinyloxy or pyrrolidinyloxy,
      where cyclopentyl, piperazinyl, piperidinyl, pyrrolidinyl, piperidinyloxy or pyrrolidinyloxy may be substituted by amino, hydroxyl, (C₁-C₃)-alkyl or benzyloxy,
   and phenyl or thienyl,
      where phenyl or thienyl may be substituted by (C₁-C₃)-alkyl, which for its part may be substituted by amino or (C₁-C₆)-alkylamino,
         and their salts, hydrates, hydrates of the salts and solvates.

Particular preference is also given to compounds of the formula (I), in which
A is a radical

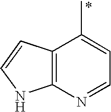

in which * represents the point of attachment to Y.

The present invention also provides compounds of the formula (I) in which
A represents a radical

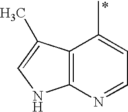

in which * represents the point of attachment to Y.

Particular preference is also given to compounds of the formula (I) in which Y is oxygen.

Particular preference is also given to compounds of the formula (I) in which R¹ is fluorine, R² is hydrogen or fluorine and R³ and R⁴ are hydrogen.

Particular preference is also given to compounds of the formula (I) in which R⁵ is hydrogen.

Particular preference is also given to compounds of the formula (I) in which the radical R⁶ is substituted by an amino or hydroxyl group and/or by a heterocycle which contains at least one nitrogen atom in the ring.

Very particular preference is given to combinations of two or more of the preferred ranges mentioned above.

The present invention also provides a process for preparing the compounds of the formula (I) which is characterized in that

either

### [A] Compounds of the Formula

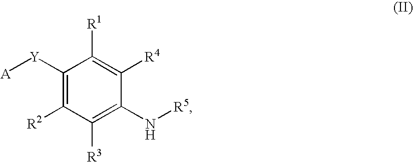 [0170] in which
A, Y, R¹, R², R³, R⁴ and R⁵ are as defined above
are reacted with compounds of the formula

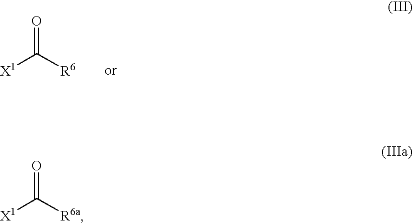

in which
R⁶ is as defined above,
R^{6a} corresponds to a radical R⁶ as defined above which, however, contains, instead of a secondary or tertiary amino group, a chlorine substituent or, instead of a free amino group, a nitro group or a protected—for example with a tert-butyloxycarbonyl group—amino group, and
X¹ represents halogen, preferably chlorine or bromine, or hydroxyl,
   and, in the case of the reaction with compounds (IIIa) in the radical R^{6a}, the chlorine substituent is subsequently substituted by an amine, the nitro group is hydrogenated to give the corresponding amino group or the protective group—for example a tert-butyloxycarbonyl protective group—is cleaved off to release the corresponding free amino group
   or

### [B] Compounds of the Formula

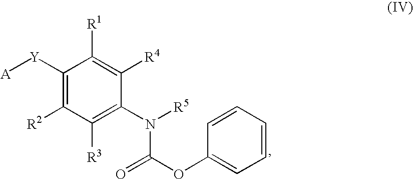 [0178] in which
A, Y, R¹, R², R³, R⁴ and R⁵ are as defined above
are reacted with compounds of the formula

H₂N—R⁸ (V),
in which
R⁸ is as defined above,
to give compounds of the formula (I).

In process step [A], if X¹ represents halogen, the reaction is generally carried out in an inert solvent, if appropriate in the presence of a base, preferably in a temperature range of from 0° C. to 50° C. at atmospheric pressure.

Inert solvents are, for example, halogenated hydrocarbons, such as methylene chloride, trichloromethane, carbon tetrachloride, trichloroethane, tetrachloroethane, 1,2-dichloroethane or trichloroethylene, ethers, such as diethyl ether, methyl tert-butyl ether, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as nitromethane, ethyl acetate, acetone, dimethylformamide, dimethylacetamide, 1,2-dimethoxyethane, 2-butanone, dimethyl sulphoxide, acetonitrile or pyridine; preference is given to tetrahydrofuran or methylene chloride.

Bases are, for example, alkali metal hydroxides, such as sodium hydroxide or potassium hydroxide, or alkali metal carbonates, such as caesium carbonate, sodium carbonate or potassium carbonate, or amides, such as lithium diisopropylamide, or other bases, such as DBU, triethylamine or diisopropylethylamine, preferably diisopropylethylamine or triethylamine.

In process step [A], if X¹ represents hydroxyl, the reaction is generally carried out in an inert solvent in the presence of customary condensing agents, if appropriate in the presence of a base, preferably in a temperature range of from room temperature to 50° C. at atmospheric pressure.

Inert solvents are, for example, halogenated hydrocarbons, such as methylene chloride, trichloromethane, carbon tetrachloride, trichloroethane, tetrachloroethane, 1,2-dichloroethane or trichloroethylene, ethers, such as diethyl ether, methyl tert-butyl ether, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as nitromethane, ethyl acetate, acetone, dimethylformamide, dimethylacetamide, 1,2-dimethoxyethane, dimethyl sulphoxide, acetonitrile or pyridine; preference is given to tetrahydrofuran, dimethylformamide or methylene chloride.

Customary condensing agents are, for example, carbodiimides, such as, for example, N,N′-diethyl-,N,N′-dipropyl-,N,N′-diisopropyl-,N,N′-dicyclohexylcarbodiimide, N-(3-dimethylaminoisopropyl)-N′-ethylcarbodiimide hydrochloride (EDC), N-cyclohexylcarbodiimide-N′-propyloxymethyl polystyrene (PS carbodiimide) or carbonyl compounds, such as carbonyldiimidazole, or 1,2-oxazolium compounds, such as 2-ethyl-5-phenyl-1,2-oxazolium 3-sulphate or 2-tert-butyl-5-methylisoxazolium perchlorate, or acylamino compounds, such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxytri-(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or 1-chloro-N,N-2-trimethylpropenylamine, or mixtures of these.

Bases are, for example, alkali metal carbonates, such as, for example, sodium carbonate or potassium carbonate or sodium bicarbonate or potassium bicarbonate, or organic bases, such as trialkylamines, for example triethylamine, N-methylmorpholine, N-methylpiperidine, 4-dimethyl-aminopyridine or diisopropylethylamine.

Preference is given to the combination of N-(3-dimethylaminoisopropyl)-N′-ethylcarbodiimide hydrochloride (EDC), 1-hydroxybenzotriazole (HOBt) and diisopropylethylamine in methylene chloride.

In process step [A], the optional substitution of the chlorine substituent by an amine, reduction of the nitro group to the corresponding amino group or removal of the amino protective group with release of the corresponding free primary or secondary amino group are in each case carried out under customary conditions familiar to the person skilled in the art. In this context, reference is made to exemplary reaction conditions in the corresponding Preparation Examples in the experimental part.

In process step [B], the conversion into compounds of the formula (I) is generally carried out in an inert solvent, preferably in a temperature range of from room temperature to the reflux temperature of the solvent at atmospheric pressure.

Inert solvents are, for example, halogenated hydrocarbons, such as methylene chloride, or ethers, such as methyl tert-butyl ether, dioxane, tetrahydrofuran, glycol dimethyl ether or diethylene glycol dimethyl ether, or other solvents, such as dimethylformamide, dimethylacetamide, dimethyl sulphoxide or acetonitrile; preference is given to dimethylformamide.

To prepare the compounds of the formula (II) from process step [A], for example, either

### [A1] Compounds of the Formula

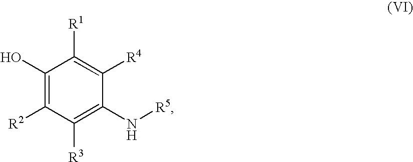 [0197] in which
R¹, R², R³ and R⁴ are as defined above
are reacted with compounds of the formula

A-X² (VII)

or

A′-X² (VIIa),
in which
A is as defined above,
A′ corresponds to a radical A as defined above which additionally contains a chlorine substituent and
X² represents halogen, preferably fluorine or chlorine, or nitro
   and, in the case of the reaction with compounds (VIIa), the chlorine substituent in the radical A′ is subsequently converted by catalytic hydrogenation into a hydrogen substituent,
   or

### [A2] Compounds of the Formula

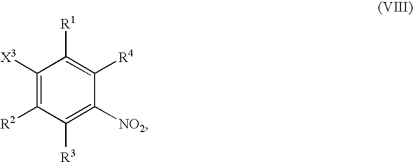 [0205] in which
R¹, R², R³ and R⁴ are as defined above, and
X³ represents halogen, preferably bromine or iodine,
are, in a first step, reacted with compounds of the formula

A-NH₂ (IX),
in which
A is as defined above,
and the nitro group is subsequently, in a second step, reduced by customary methods familiar to the person skilled in the art to the corresponding amino group, and the amino group obtained in this manner is, if appropriate, then alkylated by reaction with compounds of the formula

X⁴—R⁵ (X),
in which
R⁵ represents (C₁-C₆)-alkyl and
X⁴ represents halogen, preferably chlorine or bromine,
   by customary methods known to the person skilled in the art.

In process step [A1], the reaction is generally carried out in an inert solvent, such as, for example N,N-dimethylformamide, N-methylpyrrolidone or dimethyl sulphoxide, in the presence of a base, such as, for example, an alkali metal carbonate, such as, for example, sodium carbonate or potassium carbonate, or other bases, such as, for example, potassium tert-butoxide, potassium bis(trimethylsilyl)amide or sodium hydride, at a temperature of from 60° C. to the reflux temperature of the solvent at atmospheric pressure.

In process step [A2], the reaction is generally carried out under the conditions of the Buchwald reaction, using, for example, potassium tert-butoxide, tris(dibenzylideneacetone)dipalladium(0) [Pd₂(dba)₃] and bis(diphenylphosphino)ferrocene in toluene at a temperature of 100° C. at atmospheric pressure.

In addition, compounds of the formula (II) can be derivatized further by customary methods known to the person skilled in the art, as indicated, for example, in the Experimental Part in Examples 6A to 9A and 15A.

The compounds of the formula (IV) can be prepared, for example, by reacting compounds of the formula (II) with phenyl chloroformate according to process [A].

The compounds of the formulae (III), (IIIa), (V), (VI), (VII), (VIIa), (VIII), (IX) and (X) are known per se to the person skilled in the art or can be prepared by customary processes known from the literature.

The preparation of the compounds according to the invention can be illustrated by the synthesis scheme below.

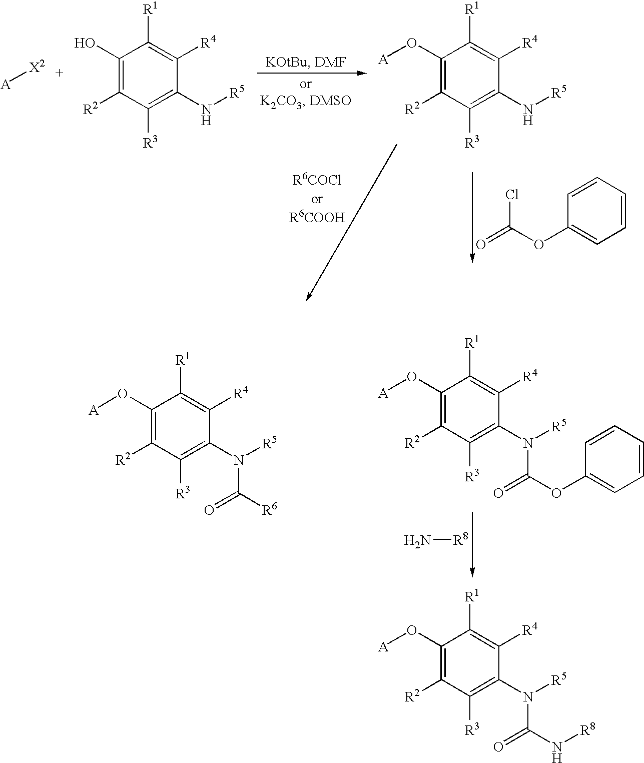

The compounds according to the invention have an unforeseeable useful spectrum of pharmacological and pharmacokinetic actions.

Accordingly, they are suitable for use as pharmaceuticals for the treatment and/or prophylaxis of diseases in humans and animals.

The pharmaceutical activity of the compounds according to the invention can be explained by their action as Rho kinase inhibitors.

The present invention also provides the use of the compounds according to the invention for the treatment of and/or prophylaxis of disorders, preferably cardiovascular disorders.

The compounds according to the invention are suitable for the prophylaxis and/or treatment of cardiovascular disorders such as, for example, hypertension and cardiac insufficiency, stable and unstable angina pectoris, disorders of peripheral and cardiac vessels, of arrhythmias, of thrombolic disorders and ischaemias, such as myocardial infarction, stroke, transitory and ischaemic attacks, obstruction of peripheral circulation, subarachnoidal haemorrhages, prevention of restenoses, such as, for example, after thrombolysis therapies, percutaneous transluminal angioplasties (PTA), percutaneous transluminal coronary angioplasties (PTCA), bypass, and for the prophylaxis and/or treatment of arteriosclerosis, Alzheimer's disease, kidney insufficiency, glaucoma, asthmatic disorders, COPD and diseases of the urogenital system, such as, for example, prostate hypertrophy, erectile dysfunction, female sexual dysfunction, osteoporosis, gastroparesis and incontinence.

The compounds according to the invention can furthermore be used for the prophylaxis and/or treatment of cancer, in particular of tumours.

In the context of the present invention, the definition of tumours includes both benign and malignant tumours and thus, for example, also benign neoplasias, dysplasias, hyperplasias, and neoplasias with metastasis formation. Further examples of tumours are carcinomas, sarcomas, carcincosarcomas, tumours of the hemopoietic organs, tumours of the nervous tissue, for example of the brain, or tumours of skin cells. In tumour formation, uncontrolled or inadequately controlled cell division occurs. The tumour can be locally restricted, but it can also infiltrate the surrounding tissue and then get lodged by the lymphatic system or by the bloodstream in a new location. There are thus primary and secondary tumours. Primary tumours are originally formed in the organ in which they are found. Secondary tumours have been lodged in another organ by metastasis formation and then spread in their new location.

The present invention also provides the use of the compounds according to the invention for the prophylaxis and/or treatment of disorders, in particular the syndromes mentioned above.

The present invention also provides the use of the compounds according to the invention for preparing a medicament for the prophylaxis and/or treatment of disorders, in particular the syndromes mentioned above.

The present invention also provides a method for the prophylaxis and/or treatment of disorders, in particular the disorders mentioned above, using a cardiovascularly effective amount of the compound according to the invention.

The present invention also provides medicaments, comprising a compound according to the invention and one or more further active compounds, in particular for the prophylaxis and/or treatment of the disorders mentioned above.

The compound according to the invention can act systemically and/or locally. For this purpose, it can be administered in a suitable manner, such as, for example, orally, parenterally, pulmonarily, nasally, sublingually, lingually, buccally, rectally, transdermally, conjunctivally, otically, as stents or as an implant.

For these administration routes, the compound according to the invention can be administered in suitable administration forms.

Suitable for oral administration are administration forms working according to the prior art, which release the compounds according to the invention rapidly and/or in modified form and which contain the compounds according to the invention in crystalline and/or amorphized and/or dissolved form, such as, for example, tablets (non-coated or coated tablets, for example coated with enteric, slowly dissolving or insoluble coats which control the release of the compounds according to the invention), tablets which decompose rapidly in the oral cavity or films/wafers, capsules, sugar-coated tablets, granules, pellets, powders, emulsions, suspensions, aerosols or solutions.

Parenteral administration can take place with circumvention of an absorption step (intravenous, intraarterial, intracardiac, intraspinal or intralumbar) or with involvement of an absorption (intramuscular, subcutaneous, intracutaneous, percutaneous or intraperitoneal). For parenteral administration, suitable administration forms are, inter alia, injection and infusion preparations in the form of solutions, suspensions, emulsions, lyophilisates and sterile powders. Suitable for the other administration routes are, for example, pharmaceutical forms for inhalation (inter alia powder inhalers, nebulizers), nasal drops/solutions, sprays; tablets or capsules to be applied lingually, sublingually or buccally, suppositories, ear and eye preparations, gyno capsules, aqueous suspensions (lotions, shake lotions), lipophilic suspensions, ointments, creams, milk, pastes, dusting powder, stents, or implants.

The compounds according to the invention can be converted into the administration forms mentioned in a manner known per se. This takes place using inert non-toxic, pharmaceutically acceptable auxiliaries. These include, inter alia, carriers (for example microcrystalline cellulose), solvents (for example liquid polyethylene glycols), emulsifiers (for example sodium dodecylsulphate), dispersants (for example polyvinylpyrrolidone), synthetic and natural biopolymers (for example albumin), stabilizers (for example antioxidants, such as ascorbic acid), colorants (for example inorganic pigments, such as iron oxides) or taste and/or odour corrigents.

The present invention also provides medicaments comprising at least one compound according to the invention, preferably together with one or more inert non-toxic, pharmaceutically suitable auxiliaries, and their use for the purposes mentioned above.

In general, it has been found to be advantageous both in human and in veterinary medicine to administer the compound according to the invention in total amounts of from about 0.01 to about 700, preferably 0.01 to 100, mg/kg of body weight per 24 hours, if appropriate in the form of a plurality of individual doses, to obtain the desired results. An individual dose contains the compound according to the invention preferably in amounts of from about 0.1 to about 80, in particular 0.1 to 30, mg/kg of body weight.

In spite of this, it may be necessary, if appropriate, to deviate from the amounts mentioned, namely depending on the body weight, the route of administration, the individual response to the active compound, the type of preparation and the time or interval at which administration takes place. Thus, in some cases it may be sufficient to use less than the abovementioned minimum amount, whereas in other cases the upper limit mentioned has to be exceeded. In the case of the administration of relatively large amounts, it may be advisable to divide these into several individual administrations over the course of the day.

The percentages in the tests and examples below are, unless indicated otherwise, percentages by weight; parts are parts by weight. Solvent ratios, dilution ratios and concentrations of liquid/liquid solutions are in each case based on the volume.

### A. EXAMPLES

### Abbreviations

TLC thin-layer chromatography
DCI direct chemical ionization (in MS)
DCM dichloromethane
DIEA N,N-diisopropylethylamine
DMA N,N-dimethylacetamide
DMF N,N-dimethylformamide
DMSO dimethyl sulphoxide
EA ethyl acetate
EI electron impact ionization (in MS)
ESI electrospray ionization (in MS)
m.p. melting point
sat. saturated
h hour
HATU O-(7-azabenzotriazol-1-yl)-N,N,N′,N′-tetramethyluronium hexafluorophosphate
HOAT 3H-[1,2,3]-triazol[4,5-b]pyridin-3-ole
HOBt 1-hydroxy-1H-benzotriazole×H₂O
HPLC high pressure, high performance liquid chromatography conc. concentrated
LC-MS liquid chromatography-coupled mass spectroscopy
LDA lithium diisopropylamide
min minutes
MPLC medium pressure, medium performance liquid chromatography
MS mass spectroscopy
NMR nuclear magnetic resonance spectroscopy
org. organic
RF reflux
R_{f} retention factor (in TLC)
RP-HPLC reverse Phase HPLC
RT room temperature
Rₜ retention time (in HPLC)
TFA trifluoroacetic acid
THF tetrahydrofuran

### HPLC, LCMS and GCMS Methods:

### Method 1 (LC/MS)

Instrument type MS: Micromass ZQ; instrument type HPLC: Waters Alliance 2795; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

### Method 2 (LC/MS)

Instrument: Micromass Quattro LCZ with HPLC Agilent Series 1100; column: Phenomenex Synergi 2μ Hydro-RP Mercury 20 mm×4 mm; mobile phase A: 1 l of water+0.5 ml of 50% strength formic acid, mobile phase B: 1 l of acetonitril+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 208-400 nm.

### Method 3 (LC/MS)

Instrument type MS: Micromass ZQ; instrument type HPLC: HP 1100 Series; UV DAD; column: Phenomenex Synergi 2μ Hydro-RP Mercury 20 mm×4 mm; mobile phase A: 1 l of water+0.5 ml of 50% strength formic acid, mobile phase B: 111 of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

### Method 4 (LC/MS)

Instrument type MS: Micromass ZQ; instrument type HPLC: Waters Alliance 2790; column: Grom-Sil 120 ODS-4 HE 50 mm×2 mm, 3.0 μm; mobile phase A: water+500 μl of 50% strength formic acid/l; mobile phase B: acetonitrile+500 μl of 50% strength formic acid/l; gradient: 0.0 min 5% B→2.0 min 40% B→4.5 min 90% B→5.5 min 90% B; oven: 45° C.; flow rate: 0.0 min 0.75 ml/min→4.5 min 0.75 ml/min 5.5 min→5.5 min 1.25 ml/min; UV detection: 210 nm.

### Method 5 (LC/MS)

Instrument: Micromass Platform LCZ with HPLC Agilent Series 1100; column: Thermo HyPURITY Aquastar 3μ 50 mm×2.1 mm; mobile phase A: 111 of water+0.5 ml of 50% strength formic acid, mobile phase B: 1 l of acetonitrile+0.5 ml of 50% strength formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

### Method 6 (LC/MS)

Instrument type MS: Micromass ZQ; instrument type HPLC: Waters Alliance 2795; column: Merck Chromolith SpeedROD RP-18e 50 mm×4.6 mm; mobile phase A: water+500, μl of 50% strength formic acid/l; mobile phase B: acetonitrile+500 μl of 50% strength formic acid/l; gradient: 0.0 min 10% B→3.0 min 95% B→4.0 min 95% B; oven: 35° C.; flow rate: 0.0 min 1.0 ml/min→3.0 min 3.0 ml/min→4.0 min 3.0 ml/min; UV detection: 210 nm.

### Method 7 (HPLC)

Instrument: HP 1100 with DAD detection; column: Kromasil RP-18, 60 mm×2 mm, 3.5 μm; mobile phase A: 5 ml of HClO₄/l of water, mobile phase B: acetonitrile; gradient: 0 min 2% B, 0.5 min 2% B, 4.5 min 90% B, 6.5 min 90% B; flow rate: 0.75 ml/min; temp.: 30° C.; UV detection: 210 nm.

### Preparative HPLC

Column: YMC Gel ODS-AQ S-5/15 μM, 250 mm×30 mm; mobile phase A: water, mobile phase B: acetonitrile; gradient: 0.00 min 30% B→3.00 min 30% B→34.0 min 95% B→38.0 min 30% B; temp.: room temperature; flow rate: 50 ml/min; UV detection.

### Starting Materials

### Example 1A

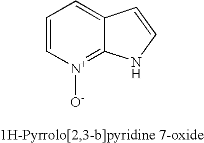

540 g (2.35 mol) of 3-chloroperbenzoic acid are dissolved in 6.11 l of dichloromethane, and water that has separated off is removed. The organic phase is dried over sodium sulphate and cooled to 0° C. A solution of 163 g (1.38 mol) of 1H-pyrrolo[2,3-b]pyridine in 1.00 l of dichloromethane is then added, and the temperature is allowed to rise to room temperature. After 2 hours, methanol is added in such a quantity that the precipitate formed is re-dissolved. The mixture is filtered through silica gel (mobile phase: dichloromethane/methanol 95:5) and the product fractions are, after concentration under high vacuum, dried.

Yield: 145 g (75% of theory)

HPLC (Method 7): Rₜ=2.02 min

MS (ESI pos.): m/z=135 (M+H)⁺, 152 (M+NH₄)⁺, 269 (2M+H)⁺

¹H-NMR (DMSO-d₆, 200 MHz): δ=6.58 (d, 1H), 7.07 (dd, 1H), 7.48 (d, 1H), 7.65 (d, 1H), 8.17 (d, 1H), 12.42-12.64 (br. s, 1H).

### Example 2A

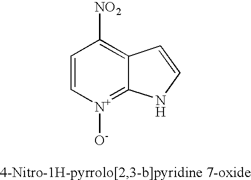

Based on the results of differential thermoanalysis, it is not recommended to carry out reactions on a scale larger than five times the amount described.

A solution of 20.0 g (149 mmol) of 1H-pyrrolo[2,3-b]pyridine 7-oxide (from Example 1A) in 160 ml of trifluoroacetic acid is cooled to room temperature. Subsequently, 69.3 ml of 65% strength nitric acid are slowly added dropwise and the mixture is stirred at room temperature for 2 h. The mixture is poured onto ice and the pH is adjusted to 8-9 using 45% sodium hydroxide solution. The precipitate is filtered off with suction and washed with water. The crude products of 4 reactions of the size described and one 13 g reaction carried out analogously are combined and purified together. The crude products are suspended in water and the pH is adjusted to 8-9 using 2N sodium hydroxide solution. After 10 min of stirring, the precipitate is filtered off with suction and dried under high vacuum.

Yield: 29.7 g (24% of theory)

HPLC (Method 7): Rₜ=3.02 min

MS (ESI pos.): m/z=180 (M+H)⁺, 197 (M+NH₄)⁺, 359 (2M+H)⁺

¹H-NMR (DMSO-d₆, 200 MHz): δ=7.03 (d, 1H), 7.80 (d, 1H), 8.03 (d, 1H), 8.31 (d, 1H), 13.22-13.41 (br. s, 1H).

### Example 3A

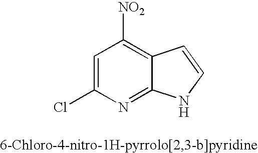

Under an atmosphere of argon, 5.00 g (27.9 mmol) of 4-nitro-1H-pyrrolo[2,3-b]pyridine 7-oxide (from Example 2A) and 11.8 ml (55.8 mmol) of hexamethyldisilazane are initially charged in 290 ml of THF. At RT, 10.8 ml (140 mmol) of methyl chloroformate are added. The solution is stirred at RT overnight. The reaction solution is filtered through a silica gel cartridge and the filtercake is washed with dichloromethane/methanol 10:1.

Yield: 2.8 g (70% of theory)

LC-MS (Method 4): Rₜ=2.74 min

MS (ESI pos.): m/z=198 (M+H)⁺

¹H-NMR (DMSO-d₆, 200 MHz): δ=7.00 (mc, 1H), 7.97 (s, 1H), 8.00 (t, 1H), 12.79 (s, 1H).

### Example 4A

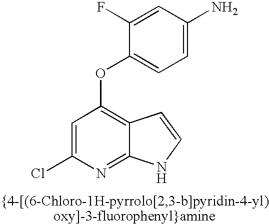

4-Amino-2-fluorophenol (0.77 g, 6.07 mmol) is dissolved in DMF. Potassium tert-butoxide (0.68 g, 6.07 mmol) is added, and the mixture is stirred at room temperature for 30 minutes. Powdered potassium carbonate (0.35 g, 2.53 mmol) and 1.00 g (5.06 mmol) of 6-chloro-4-nitro-1H-pyrrolo[2,3-b]pyridine (from Example 3A) are then added successively. The mixture is stirred at 120° C. for 12 hours. After cooling, the mixture is diluted with ethyl acetate (200 ml). The suspension is filtered off with suction through Celite® and the filtercake is washed with ethyl acetate. The solution is extracted successively with aqueous sodium bicarbonate solution and sodium chloride solution. The organic phase is dried over anhydrous magnesium sulphate and concentrated. The residue is purified by column chromatography (silica gel 60, mobile phase: dichloromethane:acetone=5:1).

Yield: 0.95 g (67% of theory)

LC-MS (Method 6): Rₜ=1.99 min

MS (ESIpos): m/z=278 (M+H)⁺

### Example 5A

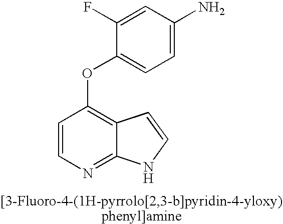

At 50° C., 3.2 g (11.5 mmol) of {4-[(6-chloro-1H-pyrrolo[2,3-b]pyridin-4-yl)oxy]-3-fluorophenyl}-amine (from Example 4A) are dissolved in ethanol. The solution is then allowed to cool to RT, and 2.45 g (2.30 mmol) of 10% palladium-on-carbon are added. The mixture is hydrogenated overnight under a hydrogen pressure of 2 bar. The palladium is then filtered off with suction through kieselguhr, the filtercake is washed with ethanol and the filtrate is concentrated.

Yield: 2.5 g (89% of theory)

LC-MS (Method 4): Rₜ=2.43 min

MS (ESI pos.): m/z=244 (M+H)⁺

¹H-NMR (DMSO-d₆, 200 MHz): δ=5.45 (mc, 2H), 6.25 (mc, 2H), 6.40-6.55 (br. 2H), 7.05 (t, 1H), 7.33 (mc, 1H), 8.25 (d, 1H), 11.69 (s, 1H).

### Example 6A

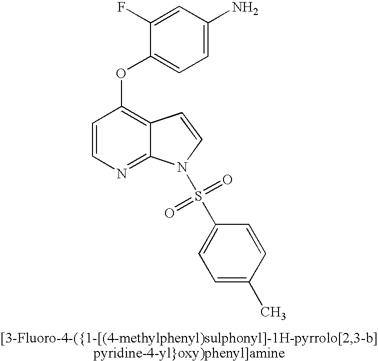

At RT, 998 mg (4.10 mmol) of [3-fluoro-4-(1H-pyrrolo[2,3-b]pyridin-4-yloxy)phenyl]amine (from Example 5A) are dissolved in 50 ml of THF, 230 mg (5.74 mmol) of sodium hydride (in THF) are added and the mixture is then stirred for one hour. Subsequently, 860 mg (4.51 mmol) of p-toluenesulphonyl chloride are added, and the reaction solution is stirred at 60° C. for another hour. The suspension is filtered through Celite®, the filtercake is washed with THF and a little dichloromethane/methanol 10:1 and the solvent is removed under reduced pressure. The residue is reacted further as crude product.

Yield: 1.65 g (86% of theory)

LC-MS (Method 1): Rₜ, =2.39 min

### Example 7A

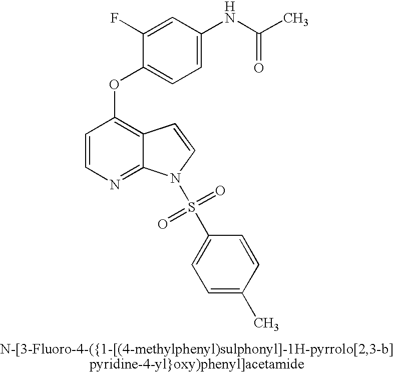

3.02 g (7.60 mmol) of [3-fluoro-4-({1-[(4-methylphenyl)sulphonyl]-1H-pyrrolo[2,3-b]pyridin-4-yl}oxy)phenyl]amine (from Example 6A) are dissolved in 30 ml of acetic anhydride, and the solution is stirred at 50° C. for one hour. Volatile components are then removed under reduced pressure and excess reagent is repeatedly removed by azeotropic distillation with toluene. The crude product is purified on a silica gel column (mobile phase: cyclohexane:ethyl acetate 1:1).

Yield: 2.04 g (58% of theory)

LC-MS (Method 3): Rₜ=2.50 min

MS (ESI pos.): m/z=440 (M+H)⁺

¹H-NMR (DMSO-d₆, 400 MHz): δ=2.07 (s, 3H), 2.35 (s, 3H), 6.55 (m, 1H), 6.66 (m, 1H), 7.34 (mc, 2H), 7.43 (d, 2H), 7.80 (m, 2H), 8.01 (d, 2H), 8.20 (d, 1H), 10.26 (s, 1H).

### Example 8A

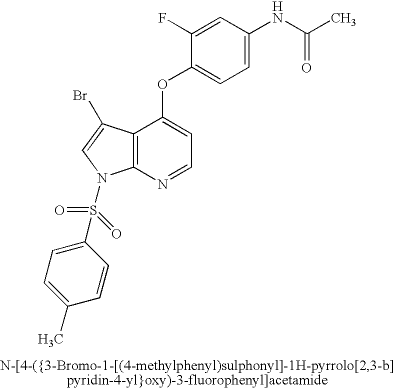

490 mg (1.11 mmol) of N-[3-fluoro-4-({1-[(4-methylphenyl)sulphonyl]-1H-pyrrolo[2,3-b]pyridin-4-yl}oxy)phenyl]acetamide (from Example 7A) are dissolved in 35 ml of dichloromethane and cooled to 0° C. 114 μl (2.23 mmol) of bromine are added. After one hour, ice and 10% strength sodium thiosulphate solution are added. After extraction with dichloromethane, the organic phase is dried over magnesium sulphate and the solvent is removed under reduced pressure. The product is purified by chromatography on silica gel (mobile phase: dichloromethane:acetone: 10:1).

Yield: 360 mg (62% of theory)

LC-MS (Method 1): Rₜ=2.50 min

¹H-NMR (DMSO-d₆, 400 MHz): δ=2.07 (s, 3H), 2.36 (s, 3H), 6.50 (m, 1H), 7.34 (mc, 2H), 7.44 (d, 2H), 7.80 (m, 1H), 8.02 (d, 2H), 8.08 (s, 1H), 8.23 (d, 1H), 10.23 (s, 1H).

### Example 9A

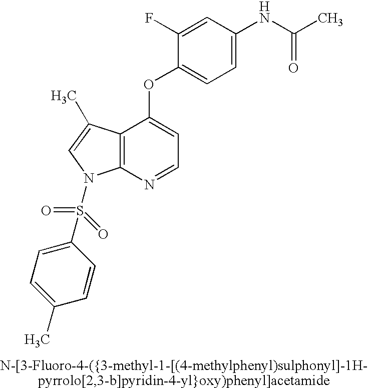

200 mg (0.39 mmol) of N-[4-({3-bromo-1-[(4-methylphenyl)sulphonyl]-1H-pyrrolo[2,3-b]pyridin-4-yl}oxy)-3-fluorophenyl]acetamide (from Example 8A) and 97 mg (1.16 mmol) of sodium bicarbonate are suspended in a mixture of dimethoxyethane (10 ml) and water (3 ml), and the mixture is degassed. 15.7 mg (0.02 mmol) of [1,1′-bis(diphenylphosphino)ferrocene]palladium(II) chloride/methylene dichloride complex and 107 μl (0.77 mmol) of trimethylboroxine are added, and the mixture is heated at 85° C. for two hours. For work-up, the reaction mixture is filtered through a silica gel Extrelut cartridge using 2 ml of dichloromethane/methanol 10:1, and the solvent is removed under reduced pressure. The residue is purified by preparative HPLC.

Yield: 83 mg (47% of theory)

LC-MS (Method 1): Rₜ=2.43 min

¹H-NMR (DMSO-d₆, 200 MHz): δ=2.08 (s, 3H), 2.35 (s, 3H), 2.39 (m, 3H), 6.40 (d, 1H), 7.34 (m, 2H), 7.41 (d, 2H), 7.62 (d, 1H), 7.78 (m, 1H), 7.95 (d, 2H), 8.14 (d, 1H), 10.22 (s, 1H).

### Example 10A

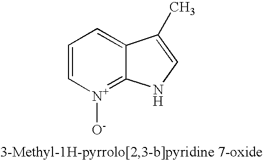

Analogously to Example 1A, the title compound is obtained by oxidation of 11.0 g (54.1 mmol) of 3-methyl-1H-pyrrolo[2,3-b]pyridine (Hands, D.; Bishop, B.; Cameron, M.; Edwards, T. S.; Cottrell, I. F.; Wright, S. H. B.; Synthesis 1996 (7), 877-882) using 24.2 g (108.2 mmol) of 3-chloroperbenzoic acid.

Yield: 5.4 g (67% of theory)

LC-MS (Method 3): Rₜ=1.19 min

MS (ESI pos.): m/z=149 (M+H)⁺

¹H-NMR (DMSO-d₆, 300 MHz): δ=2.25 (m, 3H), 7.05 (m, 1H), 7.21 (s, 1H), 7.59 (m, 1H), 8.10 (s, 1H), 12.06 (s, 1H).

### Example 11A

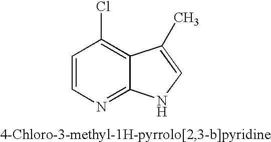

1.00 g (6.75 mmol) of 3-methyl-1H-pyrrolo[2,3-b]pyridine 7-oxide (from Example 10A) is suspended in 5 ml of phosphoryl chloride. 2 ml of chloroform are then added, and the mixture is heated at reflux temperature overnight. The mixture is allowed to cool to RT and poured into ethyl acetate/ice water. Solid sodium carbonate is then added. The phases are separated and the aqueous phase is washed with ethyl acetate. The organic phases are dried over sodium sulphate and concentrated. The residue is purified by column chromatography (silica gel 60, mobile phase: cyclohexane:methanol=4:1).

Yield: 200 mg (18% of theory)

LC-MS (Method 3): Rₜ=2.05 min

¹H-NMR (DMSO-d₆, 200 MHz): δ=2.41 (m, 3H), 7.10 (d, 1H), 7.31 (s, 1H), 8.07 (d, 1H), 12.44 (s, 1H).

### Example 12A

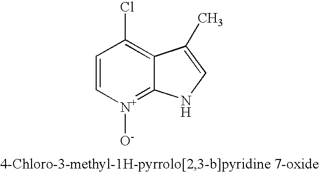

Analogously to Example 1A, the title compound is obtained by oxidation of 898 mg (5.39 mmol) of 4-chloro-3-methyl-1H-pyrrolo[2,3-b]pyridine (from Example 11A) using 2.42 g (10.78 mmol) of 3-chloroperbenzoic acid.

Yield: 688 mg (70% of theory)

LC-MS (Method 3): Rₜ=1.75 min

MS (ESI pos.): m/z=183 (M+H)⁺

¹H-NMR (DMSO-d₆, 200 MHz): δ=2.41 (m, 3H), 7.10 (d, 1H), 7.30 (s, 1H), 8.07 (d, 1H), 12.44 (s, 1H).

### Example 13A

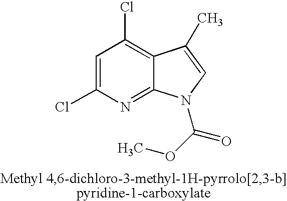

Analogously to Example 3A, the title compound is obtained from 688 mg (3.77 mmol) of 4-chloro-3-methyl-1H-pyrrolo[2,3-b]pyridine 7-oxide (from Example 12A) and 1.78 g (18.84 mmol) of methyl chloroformate and 0.61 g (3.77 mmol) of hexamethyldisilazane.

Yield: 215 mg (22% of theory)

LC-MS (Method 3): Rₜ=2.44 min

MS (ESI pos.): m/z=259 (M+H)⁺

¹H-NMR (DMSO-d₆, 200 MHz): δ=2.40 (m, 3H), 3.99 (s, 3H), 7.61 (s, 1H), 7.77 (d, 1H).

### Example 14A

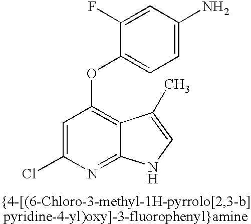

300 mg (1.16 mmol) of methyl 4,6-dichloro-3-methyl-1H-pyrrolo[2,3-b]pyridine-1-carboxylate (from Example 13A) and 320 mg (2.32 mmol) of powdered potassium carbonate are suspended in 9 ml of DMSO. The mixture is degassed and 442 mg (3.48 mmol) of 4-amino-2-fluorophenol are added. The mixture is heated at 120° C. for 4 hours. After addition of ethyl acetate, the mixture is filtered off with suction through Celite® and the filtercake is washed with ethyl acetate. The filtrate is extracted three times with saturated sodium bicarbonate solution and with saturated sodium chloride solution. The filtrate is dried over sodium sulphate and the solvent is removed under reduced pressure. The residue is purified by column chromatography (silica gel 60, mobile phase: dichloromethane:methanol=50:1).

Yield: 142 mg (42% of theory)

LC-MS (Method 3): Rₜ=2.32 min

MS (ESI pos.): m/z=292 (M+H)⁺

### Example 15A

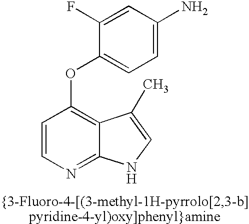

Analogously to Example 5A, the title compound is obtained by catalytic hydrogenation of 142 mg (0.49 mmol) of {4-[(6-chloro-3-methyl-1H-pyrrolo[2,3-b]pyridin-4-yl)oxy]-3-fluorophenyl}amine (from Example 14A).

Yield: 125 mg (100% of theory)

### Alternative Preparation Method:

267 mg (0.59 mmol) of N-[3-fluoro-4-({3-methyl-1-[(4-methylphenyl)sulphonyl]-1H-pyrrolo[2,3-b]pyridin-4-yl}oxy)phenyl]acetamide (from Example 9A) are dissolved in 10 ml of ethanol. 5 ml of 20% strength aqueous sodium hydroxide solution are added, and the reaction mixture is heated at 90° C. overnight. Most of the solvent is removed under reduced pressure. The residue is taken up in ethyl acetate and extracted with sodium carbonate solution. The organic phase is washed with sodium chloride solution and dried over magnesium sulphate, and the solvent is removed under reduced pressure.

Yield: 170 mg (97% of theory)

LC-MS (Method 3): Rₜ=1.52 min

¹H-NMR (DMSO-d₆, 300 MHz): δ=2.41 (s, 3H), 5.38 (s, 2H), 6.08 (d, 1H), 6.40-6.56 (m, 2H), 7.00 (t, 1H), 7.08 (s, 1H), 7.93 (d, 1H), 11.26 (s, 1H).

### Example 16A

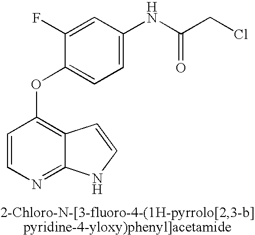

At 0° C., 0.42 ml (5.30 mmol) of chloroacetyl chloride is slowly added dropwise to a solution of 1.35 g (4.82 mmol) of [3-fluoro-4-(1H-pyrrolo[2,3-b]pyridin-4-yloxy)phenyl]amine (from Example 5A) and 1.48 ml (10.6 mmol) of triethylamine in 20 ml of dichloromethane. The mixture is allowed to stir at 0° C. for 2 hours. The reaction solution is then washed with saturated sodium carbonate solution, the organic phase is separated off and the solvent is removed under reduced pressure. The product is purified by silica gel filtration (mobile phase: ethyl acetate), giving a solid which is reacted without further purification.

Yield: 1.41 g (91% of theory)

LC-MS (Method 1): Rₜ=1.56 min

MS (ESI pos.): m/z=320, 322 (M+H)⁺

### Example 17A

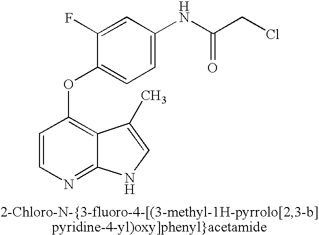

Analogously to Example 16A, the title compound is synthesized from 40 mg (0.16 mmol) of {3-fluoro-4-[(3-methyl-1H-pyrrolo[2,3-b]pyridin-4-yl)oxy]phenyl}amine (from Example 15A) and 13.6 μl (0.17 mmol) of 2-chloroacetyl chloride.

Yield: 42 mg (52% of theory)

LC-MS (Method 2): Rₜ=1.87 min

### Example 18A

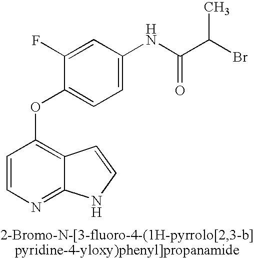

Analogously to Example 16A, the title compound is synthesized from 100 mg (0.41 mmol) of [3-fluoro-4-(1H-pyrrolo[2,3-b]pyridin-4-yloxy)phenyl]amine (from Example 5A) and 95 μl (0.90 mmol) of 2-bromopropanoyl bromide.

Yield: 173 mg (77% of theory)

LC-MS (Method 1): Rₜ=1.78 min

MS (ESI pos.): m/z=378, 380 (M+H)⁺

### Example 19A

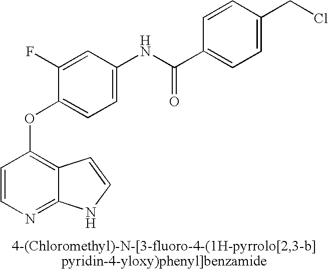

Analogously to Example 16A, the title compound is synthesized from 100 mg (0.41 mmol) of [3-fluoro-4-(1H-pyrrolo[2,3-b]pyridin-4-yloxy)phenyl]amine (from Example 5A) and 135 mg (0.71 mmol) of 4-chloromethylbenzoyl chloride.

Yield: 142 mg (65% of theory)

LC-MS (Method 1): Rₜ=2.11 min

MS (ESI pos.): m/z 396, 398 (M+H)⁺

### Example 20A

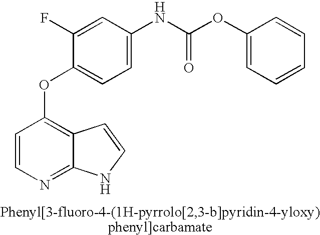

1.6 ml of a saturated solution of sodium bicarbonate in water are added to a solution of 80 mg (0.33 mmol) of [3-fluoro-4-(1H-pyrrolo[2,3-b]pyridin-4-yloxy)phenyl]amine (from Example 5A) in 3.2 ml of ethyl acetate. With vigorous stirring, 41 μl (0.33 mmol) of phenyl chloroformate are added dropwise to this suspension, and the mixture is stirred at room temperature for 2 hours. The mixture is diluted with 10 ml of ethyl acetate, the phases are separated and the organic phase is washed with 5 ml of water and saturated sodium chloride solution. The solvent is removed under reduced pressure and the product is purified by silica gel filtration (mobile phase: ethyl acetate). This gives a solid.

Yield: 102 mg (75% of theory)

LC-MS (Method 2): Rₜ=2.08 min

MS (ESI pos.): m/z=364 (M+H)⁺

### Example 21A

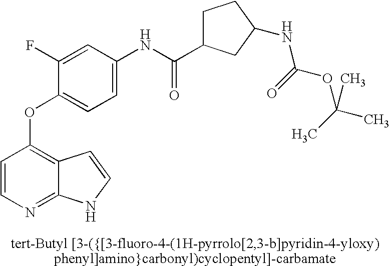

At −15° C., 69 μl (0.53 mmol) of isobutyl chloroformate are added dropwise to a solution of 122 mg (0.53 mmol) of 3-[(tert-butoxycarbonyl)amino]cyclopentanecarboxylic acid and 104 μl (0.94 mmol) of N-methylmorpholine. The mixture is allowed to stir at −15° C. for 15 minutes. At −15° C., a solution of 100 mg (0.41 mmol) of [3-fluoro-4-(1H-pyrrolo[2,3-b]pyridin-4-yloxy)-phenyl]amine (from Example 5A) in 5 ml of THF is added to the reaction. The mixture is stirred at 0° C. for 2 hours. The reaction is terminated by addition of 5 ml of water and the suspension is extracted with ethyl acetate (three times 10 ml each). The organic phase is washed with saturated sodium bicarbonate solution and dried over magnesium sulphate, and the solvent is removed under reduced pressure. The residue is dissolved in 3 ml of methanol, 22 mg (0.41 mmol) of sodium methoxide are added and the solution is stirred at room temperature for 30 minutes. The solution is purified by preparative HPLC.

Yield: 149 mg (79% of theory)

LC-MS (Method 1): Rₜ=2.07 min

MS (ESI pos.): m/z=455 (M+H)⁺

¹H-NMR (DMSO-d₆, 300 MHz): δ=1.38 (s, 9H), 1.52 (m, 1H), 1.62 (m, 1H), 1.84 (m, 3H), 2.12 (m, 1H), 2.82 (m, 1H), 3.82 (m, 1H), 6.23 (d, 1H), 6.35 (d, 1H), 6.87 (brd, 1H), 7.33 (t, 1H), 7.37 (m, 2H), 7.83 (dd, 1H), 8.06 (d, 1H), 10.19 (s, 1H), 11.75 (s, 1H).

The following compounds are prepared analogously to Example 21A:

| | | MS, HPLC, |
|---|---|---|
| Ex. No. | Structure | LC-MS, ¹H-NMR |
| 22A | 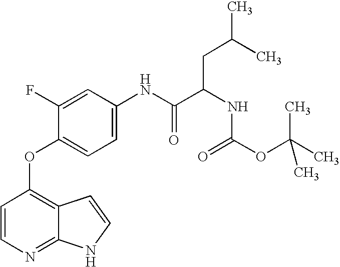 | LC-MS (Method 1): Rₜ = 2.24 minMS (ESI pos.): m/z = 457 (M + H)⁺ |
| 23A | 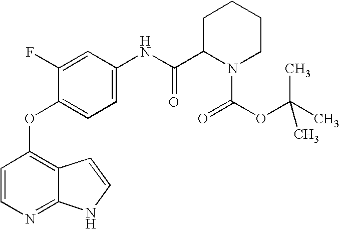 | LC-MS (Method 3): Rₜ = 2.26 minMS (ESI pos.): m/z = 455 (M + H)⁺ |
| 24A | 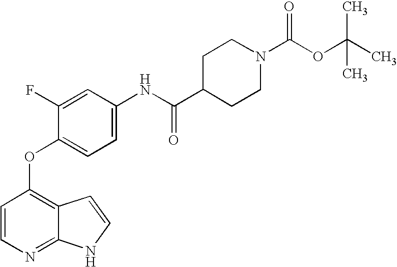 | LC-MS (Method 1): Rₜ = 2.06 minMS (ESI pos.): m/z = 455 (M + H)⁺ |
| 25A | 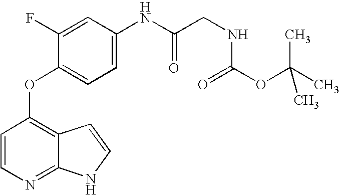 | HPLC (Method 7): Rₜ = 4.02 minMS (ESI pos.): m/z = 401 (M + H)⁺ |
| 26A | 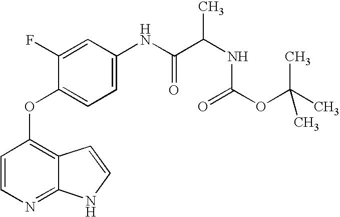 | LC-MS (Method 1): Rₜ = 1.81 minMS (ESI pos.): m/z = 415 (M + H)⁺ |
| 27A | 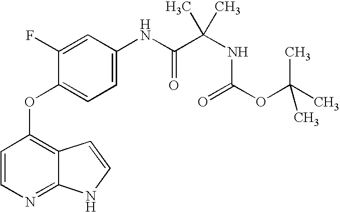 | LC-MS (Method 1): Rₜ = 1.88 minMS (ESI pos.): m/z = 429 (M + H)⁺ |
| 28A | 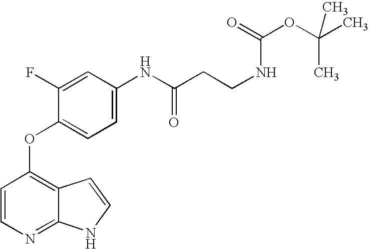 | LC-MS (Method 3): Rₜ = 1.83 minMS (ESI pos.): m/z = 415 (M + H)⁺ |
| 29A | 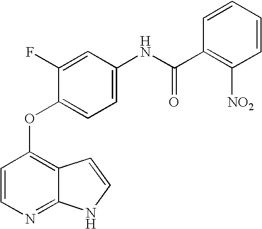 | LC-MS (Method 1): Rₜ = 1.79 minMS (ESI pos.): m/z = 393 (M + H)⁺ |
| 30A | 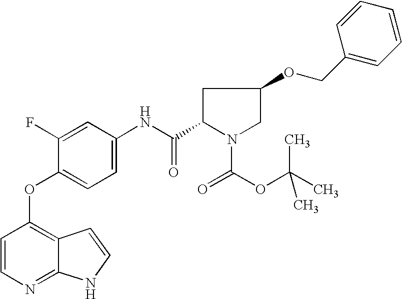 | LC-MS (Method 1): Rₜ = 2.31 minMS (ESI pos.): m/z = 547 (M + H)⁺ |
| 31A | 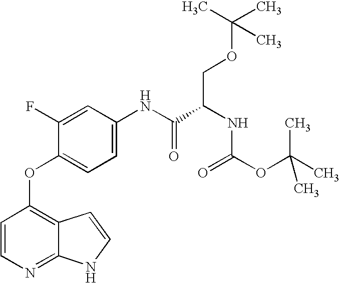 | LC-MS (Method 1): Rₜ = 2.26 minMS (ESI pos.): m/z = 487 (M + H)⁺ |

### Example 32A

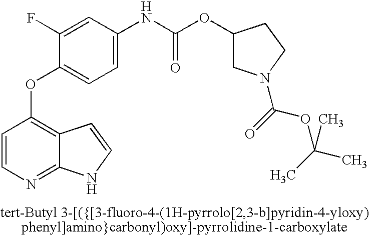

Analogously to Example 41, the title compound is synthesized from 130 mg (0.159 mmol) of phenyl[3-fluoro-4-(1H-pyrrolo[2,3-b]pyridin-4-yloxy)phenyl]carbamate (from Example 20A) and 35.7 mg (0.190 mmol) of tert-butyl 3-hydroxypyrrolidine-1-carboxylate.

Yield: 36 mg (46% of theory)

LC-MS (Method 2): Rₜ=2.16 min

MS (ESI pos.): m/z=456 (M+H)⁺

### Example 33A

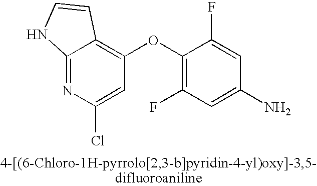

664 mg (3.36 mmol) of 6-chloro-4-nitro-1H-pyrrolo[2,3-b]pyridine (from Example 3A), 1.39 g (10.1 mmol) of powdered potassium carbonate and 877 mg (5.04 mmol) of sodium dithionite are suspended in 10 ml of DMSO. The mixture is degassed, and 915 mg (5.04 mmol) of 4-amino-2,6-difluorophenol hydrochloride are added. The mixture is heated at 120° C. for 4 hours. After addition of ethyl acetate, the mixture is filtered off with suction through Celite and the filtercake is washed with ethyl acetate. The filtrate is extracted three times with saturated sodium bicarbonate solution and with saturated sodium chloride solution. The filtrate is dried over sodium sulphate and the solvent is removed under reduced pressure. The residue is purified by column chromatography (silica gel 60, mobile phase: dichloromethane/methanol=50:1).

Yield: 356 mg (36% of theory)

LC-MS (Method 1): Rₜ=2.05 min

MS (ESI pos.): m/z=296 (M+H)⁺.

¹H-NMR (DMSO-d₆, 400 MHz): δ=5.84 (s, 2H), 6.28 (mc, 1H), 6.38-6.41 (m, 3H), 7.42 (mc, 1H), 12.02 (s, 1H).

### Example 34A

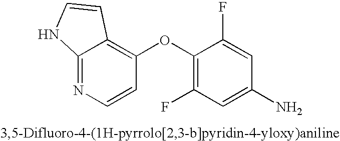

Analogously to the synthesis of 3-fluoro-4-(1H-pyrrolo[2,3-b]pyridin-4-yloxy)aniline (Example 5A), the title compound is obtained by catalytic hydrogenation from 408 mg (1.38 mmol) of 4-[(6-chloro-1H-pyrrolo[2,3-b]pyridin-4-yl)oxy]-3,5-difluoroaniline (from Example 33A).

Yield: 360 mg (100% of theory)

LC-MS (Method 1): Rₜ=1.46 min

MS (ESI pos.): m/z=262 (M+H)⁺.

¹H-NMR (DMSO-d₆, 400 MHz): δ=5.77 (br. s, 1H), 6.28 (dd, 1H), 6.34-6.40 (m, 3H), 7.37 (dd, 1H), 8.06 (d, 1H), 11.76 (br. s, 1H).

### Example 35A

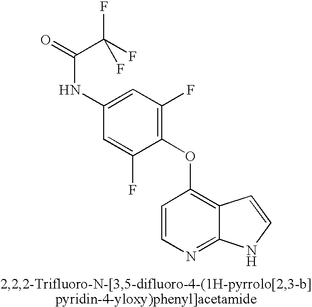

At 0° C., 0.16 ml (1.14 mmol) of trifluoroacetic anhydride is added dropwise to a solution of 200 mg (0.76 mmol) of 3,5-difluoro-4-(1H-pyrrolo[2,3-b]pyridin-4-yloxy)aniline and 0.21 ml (1.53 mmol) of triethylamine in anhydrous dichloromethane (20 ml). The mixture is allowed to stir at 0° C. for 20 min, and the reaction is terminated by dropwise addition of a saturated sodium bicarbonate solution (10 ml). The suspension is allowed to warm to RT and the phases are separated. The aqueous phase is extracted with tert-butyl methyl ether (10 ml). The combined organic phases are washed with a saturated sodium chloride solution. The organic solution is dried over magnesium sulphate and concentrated. This gives a solid which is not purified any further.

Yield: 270 mg (98% of theory)

HPLC (Method 3): Rₜ=2.21 min

MS (ESI pos.): m/z=358 (M+H)⁺.

### Example 36A

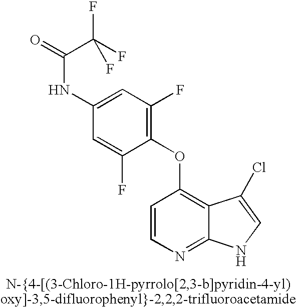

204 mg (1.54 mmol) of N-chlorosuccinimide and 50 μl of 1M aqueous hydrochloric acid are added to a solution of 250 mg (0.70 mmol) of 2,2,2-trifluoro-N-[3,5-difluoro-4-(1H-pyrrolo[2,3-b]pyridin-4-yloxy)phenyl]acetamide (from Example 35A) in anhydrous tetrahydrofuran (5 ml). The solution is allowed to stir at RT overnight. The title compound precipitates from the reaction mixture. Filtration with suction and drying gives a solid.

Yield: 90 mg (33% of theory)

HPLC (Method 3): Rₜ=2.45 min

MS (ESI pos.): m/z 392, 394 (M+H)⁺.

### Example 37A

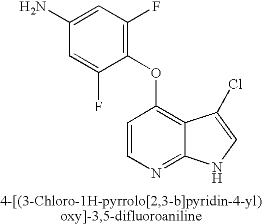

3 ml of a 1N sodium hydroxide solution are added to a solution of 90 mg (0.23 mmol) of N-{4-[(3-chloro-1H-pyrrolo[2,3-b]pyridin-4-yl)oxy]-3,5-difluorophenyl}-2,2,2-trifluoroacetamide (from Example 36A) in ethanol (5 ml). The reaction is allowed to stir overnight. The solution is extracted with tert-butyl methyl ether (two times 10 ml). The combined organic phases are washed with a saturated sodium chloride solution. The organic phase is dried over magnesium sulphate and concentrated. This gives a solid which is not purified any further.

Yield: 65 mg (96% of theory)

HPLC (Method 2): Rₜ=2.13 min

MS (ESI pos.): m/z=296, 298 (M+H)⁺.

### Example 38A

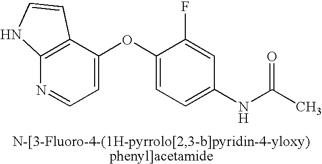

5.00 g (17.9 mmol) of 3-fluoro-4-(1H-pyrrolo[2,3-b]pyridin-4-yloxy)aniline (from Example 5A) are suspended in 100 ml of dichloromethane and cooled to 0° C. 9.97 ml (71.5 mmol) of triethylamine and 3.81 ml (53.6 mmol) of acetyl chloride are added dropwise, and the mixture is allowed to stir at 0° C. for another 2 hours. Saturated sodium bicarbonate solution is added, and the mixture is stirred at RT for 10 min. The mixture is then extracted twice with dichloromethane. Insoluble components are dissolved using a little acetone, and the mixture is diluted with dichloromethane and washed with saturated sodium chloride solution. The combined organic phases are dried over magnesium sulphate and the solvent is removed under reduced pressure. The crude product is taken up in 100 ml of methanol. 3.31 ml (17.9 mmol) of 5.4M sodium methoxide solution are added, and the mixture is allowed to stir at RT for 30 min. The mixture is concentrated and the product is purified by chromatography on silica gel (mobile phase: dichloromethane/methanol 100:1 to 100:5).

Yield: 3.92 g (77% of theory)

LC-MS (Method 3): Rₜ=1.56 min

MS (ESI pos.): m/z=286 (M+H)⁺.

¹H-NMR (DMSO-d₆, 300 MHz): δ=2.08 (s, 3H), 6.22 (dd, 1H), 6.35 (d, 1H), 7.28-7.38 (m, 3H), 7.80 (dd, 1H), 8.07 (d, 1H), 10.21 (br. s, 1H), 11.72 (br. s, 1H).

### Working Examples

### Example 1

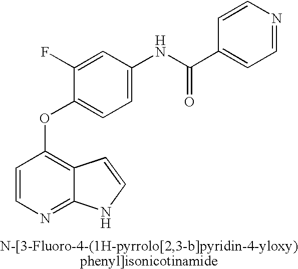

100 mg (0.41 mmol) of [3-fluoro-4-(1H-pyrrolo[2,3-b]pyridin-4-yloxy)phenyl]amine (from Example 5A) and 230 μl (1.64 mmol) of triethylamine are dissolved in 5 ml of dichloromethane. The mixture is cooled to 0° C., 175 mg (1.23 mmol) of isonicotinoyl chloride are added and the mixture is allowed to stir at room temperature for 24 h. Water is then added, the mixture is diluted with dichloromethane and extracted, the organic phase is dried over sodium sulphate and the solvent is removed under reduced pressure. The residue is suspended in 2.5 ml of methanol and, after addition of 0.09 ml (0.50 mmol) of 5.4 molar sodium methoxide solution, stirred at room temperature for 1 h. The product is purified by preparative HPLC, which gives a solid.

Yield: 70 mg (49% of theory)

LC-MS (Method 2): Rₜ=1.52 min

MS (ESI pos.): m/z=349 (M+H)⁺

¹H-NMR (DMSO-d₆, 300 MHz): δ=6.24 (dd, 1H), 6.40 (d, 1H), 7.37 (dd, 1H), 7.42 (t, 1H), 7.64 (dd, 1H), 7.87 (d, 2H), 7.97 (dd, 1H), 8.09 (d, 1H), 8.81 (d, 2H), 10.75 (s, 1H), 11.76 (s, 1H).

### Example 2

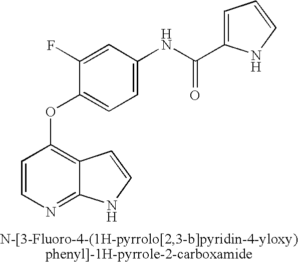

100 mg (0.41 mmol) of [3-fluoro-4-(1H-pyrrolo[2,3-b]pyridin-4-yloxy)phenyl]amine (from Example 5A), 72 μl (0.411 mmol) of diisopropylethylamine and 137 mg (1.23 mmol) of pyrrole-2-carboxylic acid are dissolved in 5 ml of dichloromethane. The mixture is cooled to 0° C., 219 mg (1.23 mmol) of N′-(3-dimethylaminopropyl)-N-ethylcarbodiimide hydrochloride (EDC) are added and the mixture is allowed to stir at room temperature for 24 h. Water is then added, the mixture is diluted with dichloromethane and extracted, the organic phase is dried over sodium sulphate and the solvent is removed under reduced pressure. The residue is purified by preparative HPLC. This gives a solid.

Yield: 46 mg (33% of theory)

LC-MS (Method 2): Rₜ=1.69 min

MS (ESI pos.): m/z=337 (M+H)⁺

### Example 3

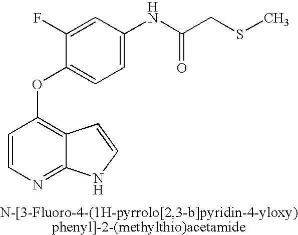

100 mg (0.36 mmol) of the hydrochloride of [3-fluoro-4-(1H-pyrrolo[2,3-b]pyridin-4-yloxy)-phenyl]amine (from Example 5A) are dissolved in a mixture of 5.0 ml of dichloromethane and 0.50 ml of pyridine. 89 mg (0.72 mmol) of methylthioacetyl chloride are added and the mixture is allowed to stir at RT for 20 h. Water is added, the mixture is diluted with dichloromethane and extracted, the organic phase is dried over sodium sulphate and the solvent is removed under reduced pressure. The residue is suspended in 2.5 ml of methanol and, after addition of 0.09 ml (0.50 mmol) of 5.4 molar sodium methoxide solution, stirred at RT for 1 h. The product is then purified by preparative HPLC.

Yield: 72 mg (61% of theory)

LC-MS (Method 3): Rₜ=1.63 min

MS (ESI pos.): m/z=332 (M+H)⁺

¹H-NMR (DMSO-d₆, 300 MHz): δ=2.18 (s, 3H), 3.30 (s, 2H), 6.24 (d, 1H), 6.37 (d, 1H), 7.3% 7.41 (m, 3H), 7.81 (d, 1H), 8.07 (d, 1H), 10.36 (br. s, 1H), 11.73 (br. s, 1H).

### Example 4

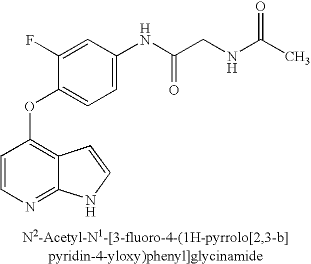

100 mg (0.41 mmol) of [3-fluoro-4-(1H-pyrrolo[2,3-b]pyridin-4-yloxy)phenyl]amine (from Example 5A) are dissolved in a mixture of 5.0 ml of dichloromethane and 0.50 ml of pyridine. 111 mg (0.82 mmol) of acetamidoacetyl chloride are added, and the mixture is allowed to stir at RT for 20 h. Water is added, the mixture is diluted with dichloromethane and extracted, the organic phase is dried over sodium sulphate and the solvent is removed under reduced pressure. The residue is suspended in 2.5 ml of methanol and, after addition of 0.09 ml (0.50 mmol) of 5.4 molar sodium methoxide solution, stirred at RT for 1 h. The product is then purified by preparative HPLC, which gives a solid.

Yield: 66 mg (47% of theory)

LC-MS (Method 1): Rₜ=1.17 min

MS (ESI pos.): m/z=343 (M+H)⁺

¹H-NMR (DMSO-d₆, 200 MHz): δ=1.90 (s, 3H), 3.89 (d, 2H), 6.21 (d, 1H), 6.36 (d, 1H), 7.31-7.42 (m, 3H), 8.06 (d, 1H), 8.26 (t, 1H), 10.29 (br. s, 1H), 11.76 (br. s, 1H).

### Example 5

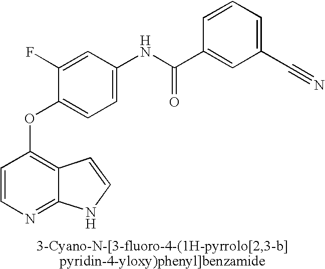

Analogously to Example 4, 93 mg (0.38 mmol) of [3-fluoro-4-(1H-pyrrolo[2,3-b]pyridin-4-yloxy)phenyl]amine (from Example 5A) are reacted with 127 mg (0.76 mmol) of 3-cyanobenzoyl chloride.

Yield: 93 mg (64% of theory)

LC-MS (Method 3): Rₜ=1.93 min

MS (ESI pos.): m/z=373 (M+H)⁺

¹H-NMR (DMSO-d₆, 200 MHz): δ=6.26 (dd, 1H), 6.40 (d, 1H), 7.38-7.48 (m, 2H), 7.60-7.68 (m, 1H), 7.79 (t, 1H), 7.99 (dd, 1H), 8.07-8.13 (m, 2H), 8.27 (d, 1H), 8.42 (s, 1H), 10.70 (s, 1H), 11.79 (s, 1H).

### Example 6

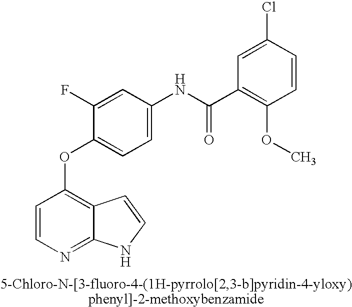

Analogously to Example 4, 100 mg (0.36 mmol) of the hydrochloride of [3-fluoro-4-(1H-pyrrolo[2,3-b]pyridin-4-yloxy)phenyl]amine (from Example 5A) are reacted with 147 mg (0.72 mmol) of 5-chloro-2-methoxybenzoyl chloride.

Yield: 74 mg (50% of theory)

LC-MS (Method 1): Rₜ=2.41 min

MS (ESI pos.): m/z=412 (M+H)⁺

¹H-NMR (DMSO-d₆, 300 MHz): δ=3.90 (s, 3H), 6.26 (s, 1H), 6.39 (d, 1H), 7.23 (d, 1H), 7.35-7.42 (m, 2H), 7.52-7.59 (m, 2H), 7.62 (s, 1H), 7.92 (d, 1H), 8.08 (d, 1H), 10.46 (s, 1H), 11.75 (s, 1H).

### Example 7

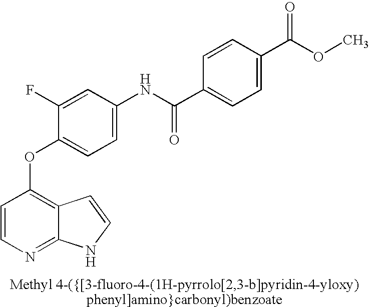

Analogously to Example 4, 100 mg (0.41 mmol) of [3-fluoro-4-(1H-pyrrolo[2,3-b]pyridin-4-yloxy)phenyl]amine (from Example 5A) are reacted with 163 mg (0.82 mmol) of monomethyl terephthaloyl chloride.

Yield: 7 mg (4% of theory)

LC-MS (Method 2): Rₜ=2.22 min

MS (ESI pos.): m/z=406 (M+H)⁺

¹H-NMR (DMSO-d₆, 400 MHz): δ=3.91 (s, 3H), 6.25 (d, 1H), 6.40 (d, 1H), 7.38 (d, 1H), 7.42 (t, 1H), 7.64-7.67 (m, 1H), 7.99 (dd, 1H), 8.08-8.14 (m, 5H), 10.72 (br. s, 1H), 11.78 (br. s, 1H).

### Example 8

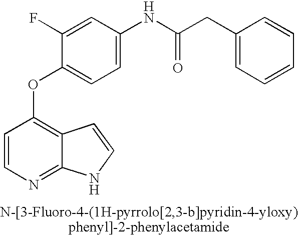

Analogously to Example 4, 100 mg (0.41 mmol) of [3-fluoro-4-(1H-pyrrolo[2,3-b]pyridin-4-yloxy)phenyl]amine (from Example 5A) are reacted with 127 mg (0.82 mmol) of phenylacetyl chloride.

Yield: 89 mg (59% of theory)

LC-MS (Method 1): Rₜ=1.90 min

MS (ESI pos.): m/z=362 (M+H)⁺

¹H-NMR (DMSO-d₆, 200 MHz): δ=3.67 (s, 2H), 6.22 (dd, 1H), 6.35 (d, 1H), 7.25-7.40 (m, 8H), 7.84 (dd, 1H), 8.06 (d, 1H), 10.48 (s, 1H), 11.76 (s, 1H).

### Example 9

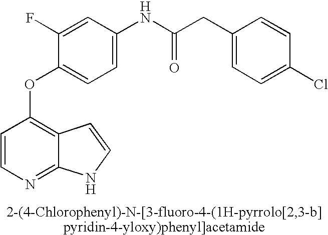

Analogously to Example 4, 100 mg (0.41 mmol) of [3-fluoro-4-(1H-pyrrolo[2,3-b]pyridin-4-yloxy)phenyl]amine (from Example 5A) are reacted with 155 mg (0.82 mmol) of 4-chlorophenylacetyl chloride.

Yield: 60 mg (35% of theory)

LC-MS (Method 1): Rₜ=2.10 min

MS (ESI pos.): m/z=396 (M+H)⁺

¹H-NMR (DMSO-d₆, 200 MHz): δ=3.69 (s, 2H), 6.22 (d, 1H), 6.35 (d, 1H), 7.29-7.44 (m, 7H), 7.77-7.85 (m, 1H), 8.06 (d, 1H), 10.50 (br. s, 1H), 11.76.

### Example 10

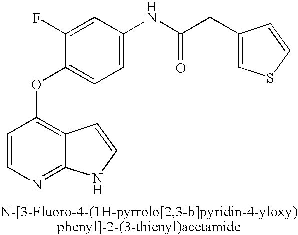

Analogously to Example 4, 100 mg (0.36 mmol) of the hydrochloride of [3-fluoro-4-(1H-pyrrolo[2,3-b]pyridin-4-yloxy)phenyl]amine (from Example 5A) are reacted with 115 mg (0.72 mmol) of thiophene-3-acetyl chloride.

Yield: 76 mg (58% of theory)

LC-MS (Method 3): Rₜ=1.90 min

MS (ESI pos.): m/z=368 (M+H)⁺

¹H-NMR (DMSO-d₆, 300 MHz): δ=3.69 (s, 2H), 6.22 (d, 1H), 6.71 (d, 1H), 7.11 (dd, 1H), 7.30-7.41 (m, 4H), 7.50 (dd, 1H), 7.82 (dd, 1H), 8.06 (d, 1H), 10.42 (br. s, 1H), 11.73 (br. s, 1H).

### Example 11

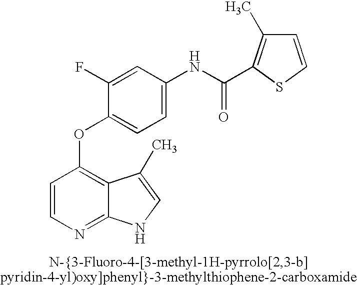

Analogously to Example 16A, the title compound is synthesized from 50 mg (0.18 mmol) of {3-fluoro-4-[(3-methyl-1H-pyrrolo[2,3-b]pyridin-4-yl)oxy]phenyl}amine (from Example 15A) and 37.3 mg (0.23 mmol) of 3-methylthiophene-2-carbonyl chloride.

Yield: 21 mg (32% of theory)

LC-MS (Method 1): Rₜ=2.02 min

MS (ESI pos.): m/z=368 (M+H)⁺

### Example 12

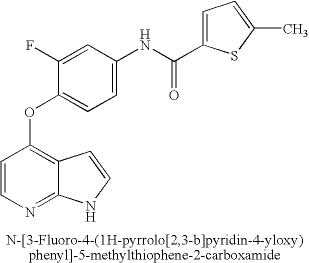

Analogously to Example 4, 100 mg (0.41 mmol) of [3-fluoro-4-(1H-pyrrolo[2,3-b]pyridin-4-yloxy)phenyl]amine (from Example 5A) are reacted with 132 mg (0.82 mmol) of 5-methylthiophene-2-carbonyl chloride.

Yield: 115 mg (76% of theory)

LC-MS (Method 1): Rₜ=2.05 min

MS (ESI pos.): m/z=368 (M+H)⁺

¹H-NMR (DMSO-d₆, 200 MHz): δ=3.40 (m, 3H), 6.25 (d, 1H), 6.40 (d, 1H), 6.95 (dd, 1H), 7.34-7.43 (m, 2H), 7.54-7.62 (m, 1H), 7.85 (d, 1H), 7.91 (dd, 1H), 8.08 (d, 1H), 10.38 (br. s, 1H), 11.77 (br. s, 1H).

### Example 13

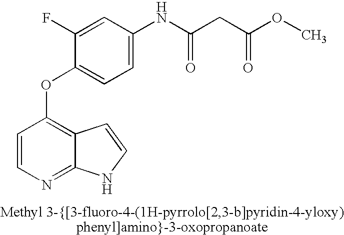

Analogously to Example 4, 100 mg (0.36 mmol) of the hydrochloride of [3-fluoro-4-(1H-pyrrolo[2,3-b]pyridin-4-yloxy)phenyl]amine (from Example 5A) are reacted with 98 mg (0.72 mmol) of methyl malonyl chloride.

Yield: 10 mg (8% of theory)

LC-MS (Method 3): Rₜ=1.51 min

MS (ESI pos.): m/z=344 (M+H)⁺

¹H-NMR (DMSO-d₆, 400 MHz): δ=6.29 (dd, 1H), 6.46 (d, 1H), 7.36-7.43 (m, 3H), 7.77-7.83 (m, 1H), 8.13 (d, 1H), 10.59 (s, 1H), 11.91 (s, 1H).

### Example 14

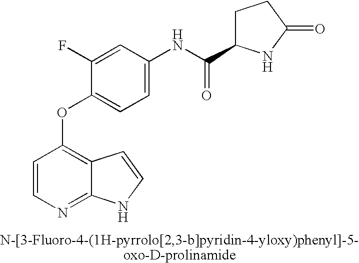

In 2.0 ml of THF, 102 mg (0.79 mmol) of D-pyroglutamic acid are initially charged at 0° C. 105 mg (0.79 mmol) of 1-chloro-N,N-2-trimethylpropenylamine are added, and the mixture is allowed to stir at this temperature for 2 h. A solution of 100 mg (0.36 mmol) of the hydrochloride of [3-fluoro-4-(1H-pyrrolo[2,3-b]pyridin-4-yloxy)phenyl]amine (from Example 5A) in a mixture of 3 ml of THF, 1 ml of DMF and 0.16 ml of 4-methylmorpholine is then added dropwise. The mixture is allowed to stir at RT for 15 h and then diluted with ethyl acetate and extracted with water. The organic phase is concentrated and the residue is dissolved in methanol, sodium methoxide solution is added and the mixture is stirred at RT for 1 h. The product is purified by preparative HPLC.

Yield: 10 mg (8% of theory)

LC-MS (Method 3): Rₜ=1.31 min

MS (ESI pos.): m/z=355 (M+H)⁺

¹H-NMR (DMSO-d₆, 300 MHz): δ=1.97-2.44 (m, 4H), 4.20 (dd, 1H), 6.22 (d, 1H), 6.37 (d, 1H), 7.32-7.46 (m, 3H), 7.83 (dd, 1H), 8.07 (d, 1H), NH (3H) not visible.

### Example 15

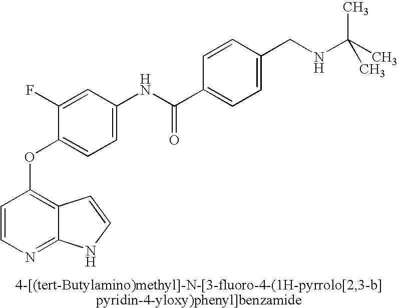

At room temperature, 73 μl (0.79 mmol) of tert-butylamine are added to a solution of 141 mg (0.23 mmol) of 2-chloro-N-[3-fluoro-4-(1H-pyrrolo[2,3-b]pyridin-4-yloxy)phenyl]benzamide (from Example 19A) in 2 ml of dimethylformamide. The mixture is allowed to stir at 40° C. for 12 hours. The solution is purified by preparative HPLC, which gives a solid.

Yield: 15 mg (14% of theory)

LC-MS (Method 2): Rₜ=1.33 min

MS (ESI pos.): m/z=433 (M+H)⁺

¹H-NMR (DMSO-d₆, 300 MHz): δ=1.40 (s, 9H), 4.19 (m, 2H), 6.37 (m, 1H), 6.57 (d, 1H), 7.47 (t, 1H), 7.49 (m, 1H), 7.76 (dd, 1H), 7.77 (d, 2H), 8.05 (dd, 1H), 8.08 (d, 2H), 8.20 (d, 1H), 9.13 (brs, 2H), 10.68 (s, 1H), 12.22 (s, 1H).

### Example 16

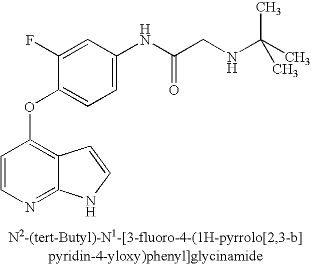

At room temperature, 140 μl (1.38 mmol) of tert-butylamine are added to a solution of 147 mg (0.46 mmol) of 2-chloro-N-[3-fluoro-4-(1H-pyrrolo[2,3-b]pyridin-4-yloxy)phenyl]acetamide (from Example 16A) in 3 ml of dimethylformamide. The mixture is allowed to stir for 12 hours and the solution is purified by preparative HPLC, which gives a solid.

Yield: 85 mg (52% of theory)

LC-MS (Method 1): Rₜ=1.03 min

MS (ESI pos.): m/z=357 (M+H)⁺

¹H-NMR (DMSO-d₆, 300 MHz): δ=1.07 (s, 9H), 6.22 (m, 1H), 6.36 (d, 1H), 7.35 (m, 2H), 7.47 (dd, 1H), 7.86 (dd, 1H), 8.06 (d, 1H), 11.73 (s, 1H).

The following compounds are prepared analogously to Example 16:

| | Starting | | |
|---|---|---|---|
| Ex. | material | | MS, HPLC, |
| No. | (Ex. No.) | Structure | LC-MS, ¹H-NMR |
| 17 | 16A | 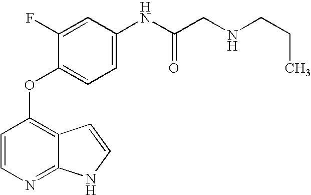 | LC-MS (Method 2): Rₜ = 1.20 minMS (ESI pos.): m/z = 345 (M + H)⁺¹H-NMR (DMSO-d₆, 300 MHz): δ = 0.89(t, 3 H), 1.45 (q, 2 H), 6.23 (m, 1 H), 6.35 (d,1 H), 7.34 (t, 1 H), 7.37 (m, 1 H), 7.46 (dd,1 H), 8.06 (d, 1 H), 7.86 (dd, 1 H), 11.75 (s,1 H). |
| 18 | 16A | 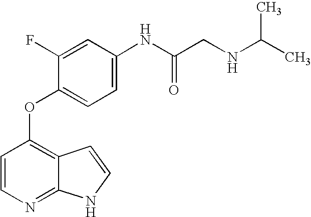 | LC-MS (Method 1): Rₜ = 0.95 minMS (ESI pos.): m/z = 343 (M + H)⁺¹H-NMR (DMSO-d₆, 300 MHz): δ = 1.01(d, 6 H), 2.74 (m, 1 H), 6.23 (m, 1 H), 6.35(d, 1 H), 7.34 (t, 1 H), 7.37 (m, 1 H), 7.46(dd, 1 H), 7.86 (dd, 1 H), 8.06 (d, 1 H),11.76 (s, 1 H). |
| 19 | 16A | 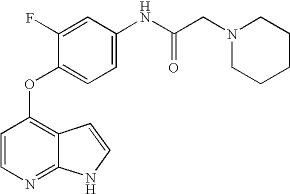 | LC-MS (Method 1): Rₜ = 0.95 minMS (ESI pos.): m/z = 343 (M + H)⁺¹H-NMR (DMSO-d₆, 300 MHz): δ = 1.41(m, 2 H), 1.57 (m, 4 H), 2.46 (m, 4 H), 3.09(s, 2 H), 6.23 (m, 1 H), 6.35 (d, 1 H), 7.34 (t,1 H), 7.37 (m, 1 H), 7.50 (dd, 1 H), 7.87 (dd,1 H), 8.06 (d, 1 H), 9.96 (s, 1 H), 11.76 (s,1 H). |
| 20 | 16A | 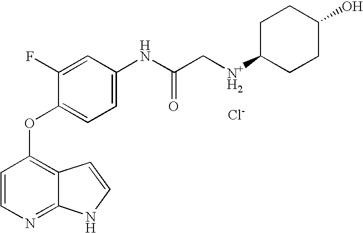 | LC-MS (Method 1): Rₜ = 0.94 minMS (ESI pos.): m/z = 399 (M + H)⁺¹H-NMR (DMSO-d₆, 300 MHz): δ = 1.20(m, 2 H), 1.43 (m, 2 H), 1.89 (m, 2 H), 2.02(m, 2 H), 3.06 (m, 1 H), 3.36 (m, 1 H), 3.99(s, 2 H), 6.30 (m, 1 H), 6.50 (d, 1 H), 7.46(m, 3 H), 7.83 (dd, 1 H), 8.16 (d, 1 H), 9.06(m, 2 H), 11.16 (s, 1 H), 12.12 (s, 1 H). |
| 21 | 16A | 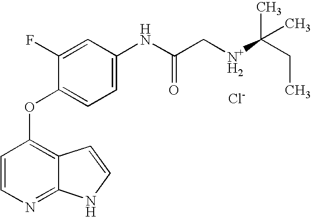 | LC-MS (Method 1): Rₜ = 0.96 minMS (ESI pos.): m/z = 373 (M + H)⁺¹H-NMR (DMSO-d₆, 300 MHz): δ = 1.26(s, 6 H), 3.06 (m, 1 H), 3.16 (s, 1 H), 3.50 (s,2 H), 6.35 (m, 1 H), 6.55 (d, 1 H), 7.48 (m,3 H), 7.84 (dd, 1 H), 8.20 (d, 1 H), 8.82 (m,2 H), 11.22 (s, 1 H), 12.28 (s, 1 H). |
| 22 | 16A | 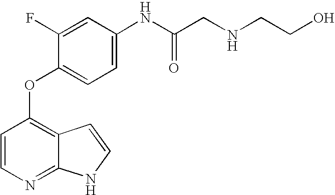 | LC-MS (Method 1): Rₜ = 0.80 minMS (ESI pos.): m/z = 345 (M + H)⁺¹H-NMR (DMSO-d₆, 200 MHz): δ = 3.71(t, 3 H), 6.34 (m, 1 H), 6.54 (d, 1 H), 7.47(m, 3 H), 7.84 (dd, 1 H), 8.19 (d, 1 H), 9.03(m, 2 H), 11.13 (s, 1 H), 12.24 (s, 1 H). |
| 23 | 17A | 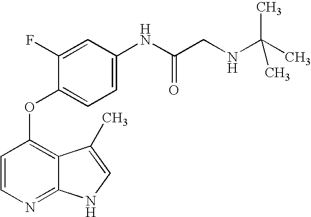 | LC-MS (Method 1): Rₜ = 1.12 min |
| 24 | 18A | 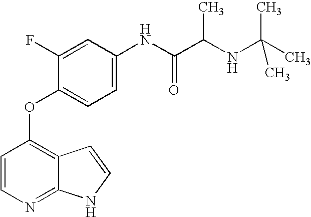 | LC-MS (Method 1): Rₜ = 1.14 minMS (ESI pos.): m/z = 371 (M + H)⁺¹H-NMR (DMSO-d₆, 300 MHz): δ = 1.05(s, 9 H), 1.23 (d, 1 H), 3.35 (m, 1 H), 6.25(m, 1 H), 6.35 (d, 1 H), 7.33 (t, 1 H), 7.36(m, 1 H), 7.48 (dd, 1 H), 7.87 (dd, 1 H), 8.05(d, 1 H), 10.20 (s, 1 H), 11.73 (s, 1 H). |

### Example 25

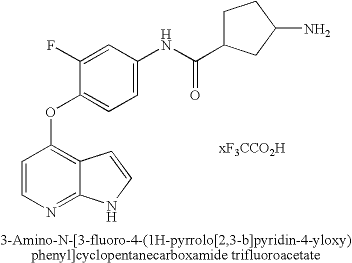

Under argon, 1 ml of trifluoroacetic acid is added to a solution of 29 mg (0.065 mmol) of tert-butyl [3-({[3-fluoro-4-(1H-pyrrolo[2,3-b]pyridin-4-yloxy)phenyl]amino}carbonyl)cyclopentyl]-carbamate (from Example 21A) in 1 ml of dichloromethane, and the mixture is allowed to stir at room temperature for 2 hours. The solvent is removed under reduced pressure and the residue is purified by preparative HPLC.

Yield: 21.4 mg (68% of theory)

LC-MS (Method 1): Rₜ=1.03 min

MS (ESI pos.): m/z=355 (M+H)⁺

### Example 26

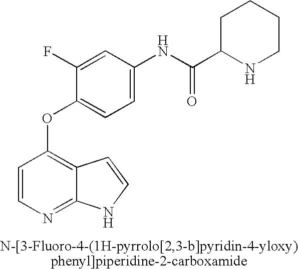

Under argon, 1 ml of trifluoroacetic acid is added to a solution of 65 mg (0.144 mmol) of tert-butyl 2-({[3-fluoro-4-(1H-pyrrolo[2,3-b]pyridin-4-yloxy)phenyl]amino}carbonyl)piperidine-1-carboxylate (from Example 23A) in 1 ml of dichloromethan, and the mixture is allowed to stir at room temperature for 2 hours. The solvent is removed under reduced pressure. The residue is dissolved in dichloromethane and washed with a saturated solution of sodium carbonate. The organic phase is dried and concentrated. The residue is purified by preparative HPLC.

Yield: 37 mg (66% of theory)

LC-MS (Method 1): Rₜ=1.02 min

MS (ESI pos.): m/z=355 (M+H)⁺

### Example 27

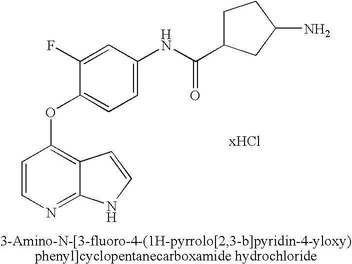

Under argon and at 0° C., 2.5 ml (10 mmol) of a 4M solution of hydrogen chloride in dioxane are added to a solution of 454 mg (1.00 mmol) of tert-butyl[3-({[3-fluoro-4-(1H-pyrrolo[2,3-b]pyridin-4-yloxy)phenyl]amino}carbonyl)cyclopentyl]carbamate (from Example 21A) in 5 ml of dioxane, and the mixture is allowed to stir at room temperature overnight. The solvent is removed under reduced pressure and the residue is purified by preparative HPLC.

Yield: 271 mg (70% of theory)

LC-MS (Method 1): Rₜ=1.03 min

MS (ESI pos.): m/z=355 (M+H)⁺

¹H-NMR (DMSO-d₆, 300 MHz): δ=1.72 (m, 1H), 1.80-2.08 (m, 4H), 2.28 (m, 1H), 3.00 (m, 1H), 6.40 (m, 1H), 6.61 (d, 1H), 7.43 (t, 1H), 7.51 (m, 2H), 7.93 (dd, 1H), 8.10 (br. s, 3H), 8.24 (d, 1H), 10.66 (s, 1H), 12.47 (s, 1H).

The following compounds are prepared analogously to Example 27:

| | Starting | | |
|---|---|---|---|
| Ex. | material | | MS, HPLC, |
| No. | (Ex.-No.) | Structure | LC-MS, ¹H-NMR |
| 28 | 22A | 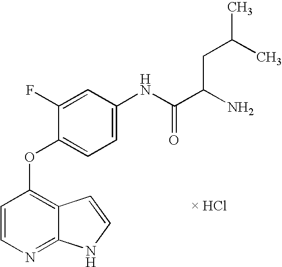 | LC-MS (Method 1): Rₜ = 1.24 minMS (ESI pos.): m/z = 357 (M + H)⁺ |
| 29 | 23A | 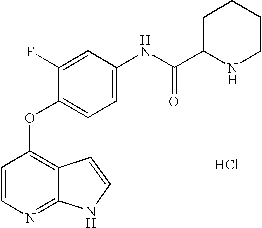 | LC-MS (Method 1): Rₜ = 1.03 minMS (ESI pos.): m/z = 355 (M + H)⁺¹H-NMR (DMSO-d₆, 300 MHz): δ = 1.45-1.89 (m, 5 H), 2.31 (m, 1 H), 2.97 (m, 1 H),3.29 (m, 1 H), 6.40 (m, 1 H), 6.61 (d, 1 H),7.48 (t, 1 H), 7.51 (m, 1 H), 7.56 (dd, 1 H),7.88 (dd, 1 H), 8.22 (d, 1 H), 8.85 (m, 1 H),9.40 (m, 1 H), 11.36 (s, 1 H), 12.38 (s, 1 H). |
| 30 | 24A | 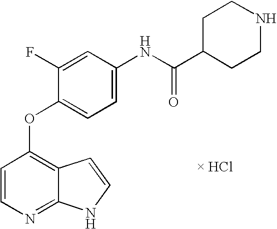 | LC-MS (Method 1): Rₜ = 0.99 minMS (ESI pos.): m/z = 355 (M + H)⁺¹H-NMR (DMSO-d₆, 200 MHz): δ = 1.71-2.09 (m, 4 H), 2.31 (m, 1 H), 2.72 (m, 1 H),2.92 (m, 2 H), 3.34 (m, 2 H), 6.44 (m, 1 H),6.62 (d, 1 H), 7.44 (t, 1 H), 7.51 (m, 2 H),7.92 (dd, 1 H), 8.25 (d, 1 H), 8.72 (m, 1 H),9.06 (m, 1 H), 10.70 (s, 1 H), 12.54 (s, 1 H). |
| 31 | 25A | 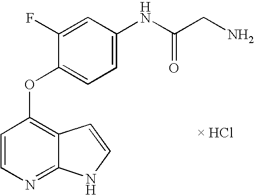 | LC-MS (Method 5): Rₜ = 2.26 minMS (ESI pos.): m/z 301 (M + H)⁺ |
| 32 | 26A | 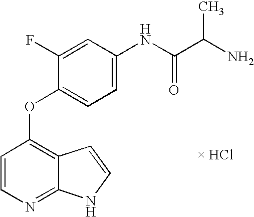 | LC-MS (Method 1): Rₜ = 0.87 minMS (ESI pos.): m/z = 315 (M + H)⁺¹H-NMR (DMSO-d₆, 300 MHz): δ = 1.51(d, 3 H), 4.14 (m, 1 H), 6.43 (m, 1 H), 6.67(d, 1 H), 7.51 (t, 1 H), 7.54 (m, 1 H), 7.59(dd, 1 H), 7.91 (dd, 1 H), 8.26 (d, 1 H), 8.41(d, 3 H), 11.42 (s, 1 H), 12.57 (s, 1 H). |
| 33 | 27A | 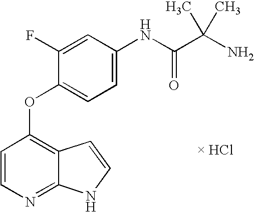 | LC-MS (Method 6): Rₜ = 1.23 minMS (ESI pos.): m/z = 329 (M + H)⁺¹H-NMR (DMSO-d₆, 300 MHz): δ = 1.68(s, 6 H), 6.41 (m, 1 H), 6.62 (d, 1 H), 7.49 (t,1 H), 7.52 (m, 1 H), 7.71 (dd, 1 H), 7.97 (dd,1 H), 8.23 (d, 1 H), 8.49 (s, 3 H), 10.74 (s,1 H), 12.43 (s, 1 H). |
| 34 | 28A | 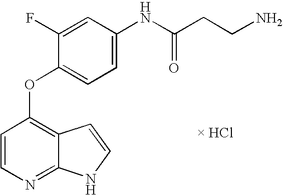 | LC-MS (Method 5): Rₜ = 2.38 minMS (ESI pos.): m/z = 315 (M + H)⁺¹H-NMR (DMSO-d₆, 300 MHz): δ = 2.42(t, 2 H), 2.86 (t, 2 H), 6.22 (d, 1 H), 6.35 (d,1 H), 7.32 (t, 1 H), 7.35 (m, 1 H), 7.37 (dd,1 H), 7.83 (dd, 1 H), 8.06 (d, 1 H), 8.49 (s,3 H), 11.73 (s, 1 H). |
| 35 | 30A | 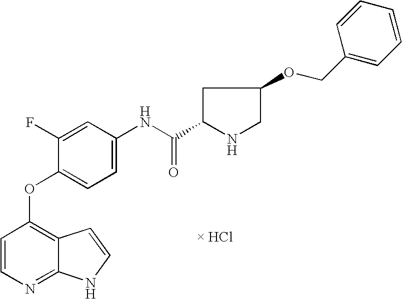 | LC-MS (Method 6): Rₜ = 1.69 minMS (ESI pos.): m/z = 447 (M + H)⁺¹H-NMR (DMSO-d₆, 200 MHz): δ = 2.09(m, 1 H), 2.77 (m, 1 H), 3.48 (m, 2 H), 4.39(m, 1 H), 4.57 (m, 3 H), 6.35 (d, 1 H), 6.55(d, 1 H), 7.25-7.45 (m, 5 H), 7.49 (m, 3 H),7.87 (dd, 1 H), 8.20 (d, 1 H), 8.93 (m, 1 H),10.09 (m, 1 H), 11.37 (s, 1 H), 12.26 (s, 1 H). |
| 36 | 31A | 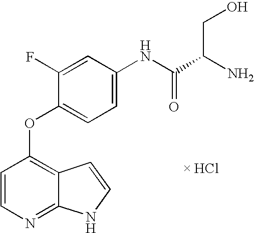 | LC-MS (Method 6): Rₜ = 1.13 minMS (ESI pos.): m/z = 331 (M + H)⁺¹H-NMR (DMSO-d₆, 300 MHz): δ = 4.11(m, 2 H), 6.41 (m, 1 H), 6.63 (d, 1 H), 7.49(t, 1 H), 7.52 (m, 1 H), 7.57 (dd, 1 H), 7.90(dd, 1 H), 8.24 (d, 1 H), 8.37 (d, 3 H), 11.33(s, 1 H), 12.48 (s, 1 H). |

### Example 37

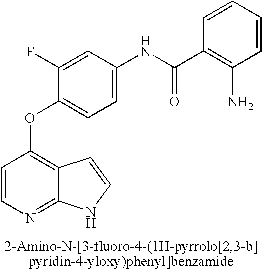

Platinum (IV) oxide is added to a solution of 50 mg (0.12 mmol) of N-[3-fluoro-4-(1H-pyrrolo[2,3-b]pyridin-4-yloxy)phenyl]-2-nitrobenzamide (from Example 29A) in 5 ml of ethanol, and the mixture is stirred at room temperature and under a hydrogen atmosphere overnight. The solid is filtered off and the solvent is removed under reduced pressure. The product is purified by preparative HPLC.

Yield: 24 mg (50% of theory)

LC-MS (Method 1): Rₜ=1.86 min

MS (ESI pos.): m/z=363 (M+H)⁺

¹H-NMR (DMSO-d₆, 200 MHz): δ=6.26 (m, 1H), 6.37 (m, 3H), 6.61 (t, 1H), 6.67 (d, 1H), 7.22 (t, 1H), 7.37 (m, 2H), 7.62 (t, 2H), 7.92 (dd, 1H), 8.07 (d, 1H), 10.26 (s, 1H), 11.77 (s, 1H).

### Example 38

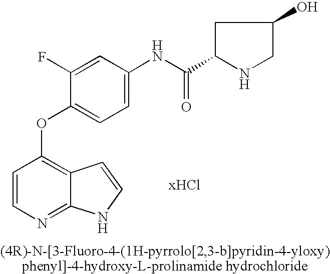

10 mg of 10% palladium-on-carbon are added to a solution of 114 mg (0.23 mmol) of (4R)-4-(benzyloxy)-N-[3-fluoro-4-(1H-pyrrolo[2,3-b]-pyridin-4-yloxy)phenyl]-L-prolinamide hydrochloride (from Example 35) in 5 ml of ethanol, and the mixture is stirred at room temperature and under an atmosphere of hydrogen overnight. The solid is filtered off and the solvent is removed under reduced pressure. The product is purified by preparative HPLC.

Yield: 14 mg (15% of theory)

LC-MS (Method 5): Rₜ=1.16 min

MS (ESI pos.): m/z=357 (M+H)⁺

¹H-NMR (DMSO-d₆, 300 MHz): δ=1.80 (m, 1H), 2.02 (dd, 1H), 2.80 (m, 1H), 2.92 (dd, 2H), 3.89 (t, 1H), 4.22 (s, 1H), 4.70 (d, 1H), 6.21 (d, 1H), 6.35 (d, 1H), 7.32 (t, 1H), 7.35 (d, 1H), 7.54 (dd, 1H), 7.90 (dd, 1H), 8.06 (d, 1H), 10.19 (m, 1H), 11.73 (s, 1H).

### Example 39

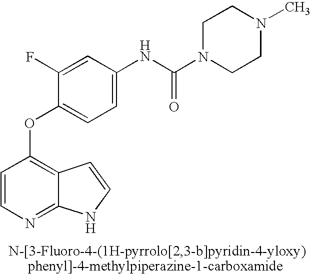

31 μl (0.27 mmol) of 1-methylpiperazine are added to a solution of 164 mg (61% pure, 0.27 mmol) of phenyl[3-fluoro-4-(1H-pyrrolo[2,3-b]pyridin-4-yloxy)phenyl]carbamate (from Example 20A) in 2 ml of DMF, and the mixture is allowed to stir at 60° C. for 3 hours. The solution is purified by preparative HPLC.

Yield: 57 mg (55% of theory)

LC-MS (Method 5): Rₜ=2.44 min

MS (ESI pos.): m/z=370 (M+H)⁺

¹H-NMR (DMSO-d₆, 300 MHz): δ=2.78 (s, 3H), 3.05 (m, 2H), 3.30 (t, 2H), 3.43 (d, 2H), 4.31 (d, 2H), 6.41 (d, 1H), 6.62 (d, 1H), 7.38 (t, 1H), 7.44 (dd, 1H), 7.53 (m, 1H), 7.75 (dd, 1H), 8.26 (d, 1H), 9.33 (s, 1H), 10.94 (s, 1H), 12.51 (s, 1H).

### Example 40

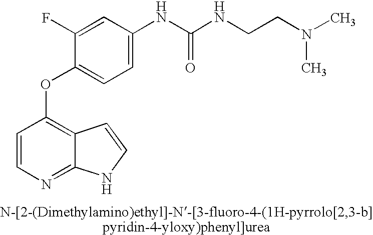

38 μl (0.367 mmol) of N,N-dimethylethylenediamine are added to a solution of 300 mg (44% pure,

0.367 mmol) of phenyl[3-fluoro-4-(1H-pyrrolo[2,3-b]pyridin-4-yloxy)phenyl]carbamate (from Example 20A) in 2 ml of DMF, and the mixture is allowed to stir at 60° C. for 3 hours. The solution is purified by preparative HPLC.

Yield: 122 mg (88% of theory)

LC-MS (Method 1): Rₜ=0.92 min

MS (ESI pos.): m/z=358 (M+H)⁺

¹H-NMR (DMSO-d₆, 300 MHz): δ=2.17 (s, 6H), 2.33 (t, 2H), 3.19 (m, 2H), 6.15 (t, 1H), 6.22 (dd, 1H), 6.33 (d, 1H), 7.08 (m, 1H), 7.24 (t, 1H), 7.34 (dd, 1H), 7.53 (m, 1H), 7.65 (dd, 1H), 8.05 (d, 1H), 8.94 (s, 1H), 11.70 (s, 1H).

### Example 41

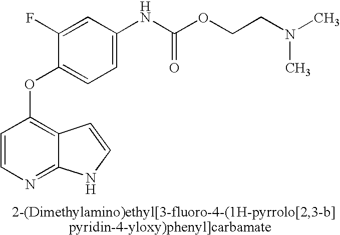

0.58 ml (0.29 mmol) of potassium hexamethyldisilazide (0.5M in THF) is added to a solution of 29 μl (0.29 mmol) of N,N-dimethylethanolamine in 2 ml of anhydrous THF. A solution of 100 mg (88% pure, 0.24 mmol) of phenyl[3-fluoro-4-(1H-pyrrolo[2,3-b]pyridin-4-yloxy)phenyl]carbamate (from Example 20A) in 2 ml of anhydrous THF is added dropwise, and the mixture is allowed to stir at RT overnight. Water is added, the mixture is extracted three times with ethyl acetate, the extracts are dried over magnesium sulphate and the solvent is removed under reduced pressure. The residue is purified by preparative HPLC.

Yield: 19 mg (20% of theory)

LC-MS (Method 1): Rₜ=1.02 min

MS (ESI pos.): m/z=359 (M+H)⁺

¹H-NMR (DMSO-d₆, 300 MHz): δ=2.20 (s, 6H), 2.53 (t, 2H), 4.19 (m, 2H), 6.22 (dd, 1H), 6.34 (d, 1H), 7.31 (m, 2H), 7.35 (dd, 1H), 7.53 (m, 1H), 7.59 (dd, 1H), 8.05 (d, 1H), 9.98 (s, 1H), 11.71 (s, 1H).

### Example 42

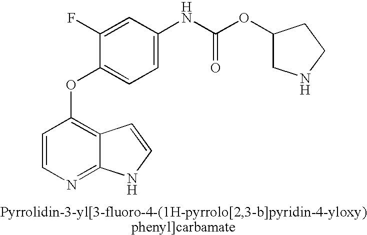

Analogously to Example 26, the title compound is synthesized from 36 mg (0.080 mmol) of tert-butyl 3-[({[3-fluoro-4-(1H-pyrrolo[2,3-b]pyridin-4-yloxy)phenyl]amino}carbonyl)oxy]pyrrolidine-1-carboxylate (from Example 32A).

Yield: 6.7 mg (33% of theory)

LC-MS (Method 1): Rₜ=0.95 min

MS (ESI pos.): m/z=357 (M+H)+

### Example 43

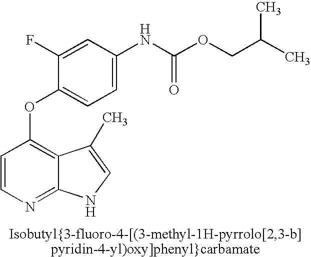

200 mg (0.77 mmol) of {3-fluoro-4-[(3-methyl-1H-pyrrolo[2,3-b]pyridin-4-yl)oxy]phenyl}amine (from Example 15A) are suspended in 5 ml of dichloromethane and cooled to 0° C. 110 μl (0.77 mmol) of triethylamine and 1.08 ml (1.16 mmol) of isobutyl chloroformate are added dropwise and the mixture is allowed to stir at 0° C. for another 2 hours. Saturated sodium bicarbonate solution is added and the mixture is allowed to stir at RT for 10 min. The mixture is then extracted twice with dichloromethane, and the organic solution is washed with saturated sodium chloride solution. The combined organic phases are dried over magnesium sulphate and the solvent is removed under reduced pressure. The crude product is taken up in 8 ml of methanol. 0.8 ml (0.8 mmol) of 1M sodium methoxide solution is added and the mixture is allowed to stir at RT for 30 min. The mixture is concentrated and the product is purified by preparative HPLC.

Yield: 200 mg (72% of theory)

LC-MS (Method 2): Rₜ=2.41 min

MS (ESI pos.): m/z=358 (M+H)⁺.

¹H-NMR (DMSO-d₆, 300 MHz): δ=0.94 (d, 6H), 1.94 (m, 1H), 3.90 (d, 2H), 6.13 (d, 1H), 7.12 (s, 1H), 7.29 (m, 2H), 7.60 (dd, 1H), 7.97 (d, 1H), 9.91 (s, 1H), 11.34 (br. s, 1H).

### Example 44

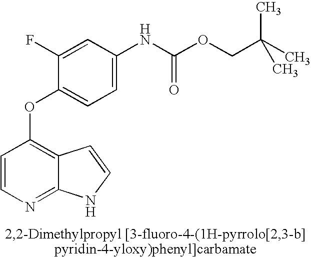

100 mg (0.41 mmol) of [3-fluoro-4-(1H-pyrrolo[2,3-b]pyridin-4-yloxy)phenyl]amine (from Example 5A) and 660 μl (6.62 mmol) of pyridine are dissolved in dichloromethane (5 ml). The mixture is cooled to 0° C., 75 mg (0.49 mmol) of neopentyl chloroformate are added and the mixture is allowed to stir at RT for 24 h. Water is then added, the mixture is diluted with dichloromethane and extracted, the organic phase is dried over sodium sulphate and the solvent is removed under reduced pressure. The residue is suspended in 2.5 ml of methanol and, after addition of 0.09 ml (0.50 mmol) of 5.4 molar sodium methoxide solution, stirred at RT for 1 h. The product is purified by preparative HPLC, which gives a solid.

Yield: 53 mg (43% of theory)

LC-MS (Method 2): Rₜ=2.45 min

MS (ESI pos.): m/z=358 (M+H)⁺

¹H-NMR (DMSO-d₆, 300 MHz): δ=0.95 (s, 9H), 3.83 (s, 2H), 6.24 (dd, 1H), 6.33 (d, 1H), 7.35 (m, 3H), 7.62 (dd, 1H), 8.06 (dd, 1H), 9.94 (s, 1H), 11.76 (s, 1H).

The following compounds are prepared analogously to the given examples:

| Ex. | | |
|---|---|---|
| No. | Structure | LC-MS |
| 45 | 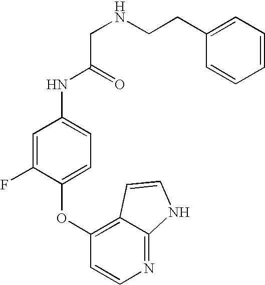 | LC-MS (Method 2): Rₜ = 1.40 minMS (ESI pos.): m/z = 405 (M + H)⁺ |
| 46 | 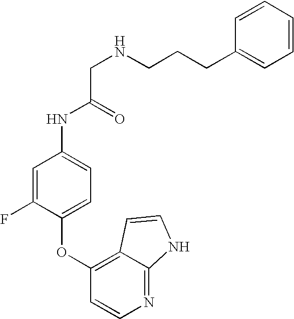 | LC-MS (Method 2): Rₜ = 1.50 minMS (ESI pos.): m/z = 419 (M + H)⁺ |
| 47 | 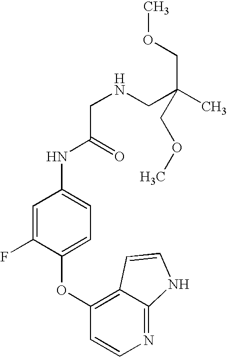 | LC-MS (Method 2): Rₜ = 1.40 minMS (ESI pos.): m/z 431 (M + H)⁺ |
| 48 | 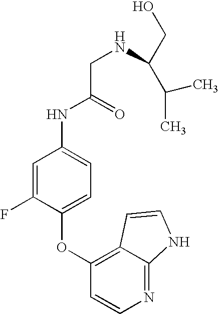 | LC-MS (Method 2): Rₜ = 1.20 minMS (ESI pos.): m/z = 387 (M + H)⁺ |
| 49 | 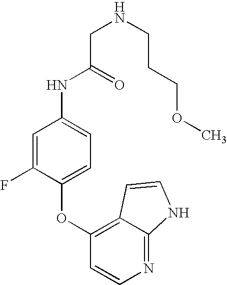 | LC-MS (Method 2): Rₜ = 1.10 minMS (ESI pos.): m/z = 373 (M + H)⁺ |
| 50 | 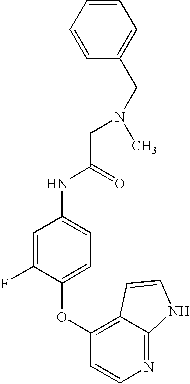 | LC-MS (Method 2): Rₜ = 1.40 minMS (ESI pos.): m/z = 405 (M + H)⁺ |
| 51 | 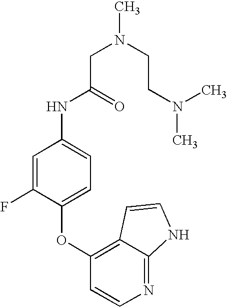 | LC-MS (Method 2): Rₜ = 1.00 minMS (ESI pos.): m/z = 386 (M + H)⁺ |
| 52 | 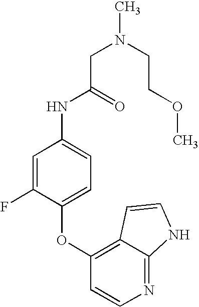 | LC-MS (Method 2): Rₜ = 1.10 minMS (ESI pos.): m/z = 373 (M + H)⁺ |

### Example 53

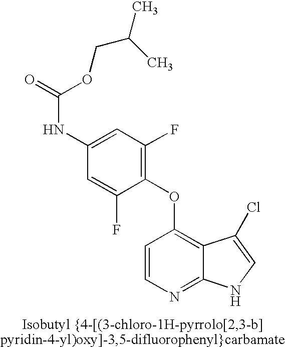

200 mg (0.50 mmol) of 4-[(3-chloro-1H-pyrrolo[2,3-b]pyridin-4-yl)oxy]-3,5-difluoroaniline (from Example 37A) are suspended in 5 ml of dichloromethane and cooled to 0° C. 70 μl (0.50 mmol) of triethylamine and 0.71 ml (0.75 mmol) of isobutyl chloroformate are added dropwise and the mixture is allowed to stir at 0° C. for another 2 hours. Saturated sodium bicarbonate solution is added, and the mixture is allowed to stir at RT for 10 min. The mixture is then extracted twice with dichloromethane and the organic solution is washed with saturated sodium chloride solution. The combined organic phases are dried over magnesium sulphate and the solvent is removed under reduced pressure. The crude product is taken up in 6 ml of methanol. 0.6 ml (0.6 mmol) of 1M sodium methoxide solution is added, and the mixture is allowed to stir at RT for 30 min. The mixture is concentrated and the product is purified by preparative HPLC.

Yield: 174 mg (65% of theory)

LC-MS (Method 3): Rₜ=2.82 min

MS (ESI pos.): m/z=396, 398 (M+H)⁺.

¹H-NMR (DMSO-d₆, 300 MHz): δ=0.94 (d, 6H), 1.94 (m, 1H), 3.92 (d, 2H), 6.34 (d, 1H), 7.42 (d, 2H), 7.61 (d, 1H), 8.11 (d, 1H), 10.18 (s, 1H), 12.13 (br. s, 1H).

### B. ASSESSMENT OF THE PHYSIOLOGICAL ACTIVITY

The inhibition of the enzyme is investigated in an in vitro assay with recombinant Rho kinase II. The vessel-relaxing action is determined using phenylephrin-induced contractions of isolated rings of the saphenous artery of rabbits. The suitability of the compounds according to the invention for treating cardiovascular disorders can be demonstrated by examining the hypotensive effect on anaesthetized rats.

### Inhibition of Recombinant Rho Kinase II (ROKα)

The activity of Rho kinase is determined by the uptake of ³³P phosphate into a substrate peptide. To this end, commercially available Rho kinase II (Upstate Biotechnology) is pre-incubated at 37° C. in the presence of the S6 phosphate-acceptor peptide with the test substances or a solvent control for 10 min. The kinase reaction is then started by addition of ³³P-labelled ATP. After 20 min at 37° C., the reaction is stopped by addition of H₃PO₄. Aliquots are pipetted onto filters and the filters are washed and then covered with scintillator. The radioactivity of the ³³P-labelled peptides bound to the filter is measured in a Micro-Beta counter. The IC₅₀ value corresponds to the concentration of a test substance at which the Rho-kinase-catalysed uptake of ³³P into the peptide is inhibited by 50%, compared to a solvent control. The experimental data are summarized in the table below.

| Example No. | IC₅₀ (nM) |
|---|---|
| 15 | 49 |
| 23 | 12 |
| 40 | 27 |
| 41 | 20 |

### Vessel-Relaxing Action In Vitro

Individual 3-mm-wide rings of the isolated saphenous artery of rabbits are introduced into 5 ml organ baths with Krebs-Henseleit solution (37° C., gassed with carbogen). The vessel tone is monitored isometrically and registered. Contractions are induced by addition of 3×10⁻⁸ g of phenylephrin/ml, which is washed out again after 4 min. After a number of control cycles, the rings are pre-incubated with the substance to be examined, with the dosage being increased for each further cycle, and the subsequent contraction is compared to the intensity of the last control contraction. The concentration required to reduce the intensity of the control value by 50% (IC₅₀) is calculated.

### Measurement of Blood Pressure in Anaesthetized Rats

Male Wistar rats of a body weight of 300-350 g are anaesthetized with thiopental (100 mg/kg i.p.). Following tracheotomy, a catheter is introduced into the femoral artery to measure the blood pressure. The substances to be tested are administered as solutions, either orally via a stomach tube or intravenously via the femoral vein.

### C. WORKING EXAMPLES FOR PHARMACEUTICAL COMPOSITIONS

The compounds according to the invention can be converted into pharmaceutical preparations as follows:

### Tablet:

### Composition:

100 mg of the compound from Example 1, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg. Diameter 8 mm, spherical radius 12 mm.

### Preparation:

The mixture of inventive compound, lactose and starch is granulated with a 5% strength solution (w/w) of the PVP in water. After drying, the granules are mixed for 5 min with the magnesium stearate. This mixture is compacted in a conventional tablet press (dimensions of the tablet: see above). The standard value used for compacting is a compaction force of 15 kN.

### Suspension for Oral Administration:

### Composition:

1000 mg of the compound from Example 1, 1000 mg of ethanol (96%), 400 mg of Rhodigel (xanthan gum from FMC, Pennsylvania, USA) and 99 g of water.

A single dose of 100 mg of the compound according to the invention corresponds to 10 ml of oral suspension.

### Preparation:

The Rhodigel is suspended in ethanol and the compound according to the invention is added to the suspension. The water is added with stirring. The mixture is stirred for about 6 h until the Rhodigel is completely swollen.

1. Compound of the formula 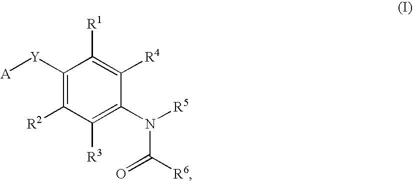
in which
A represents a radical 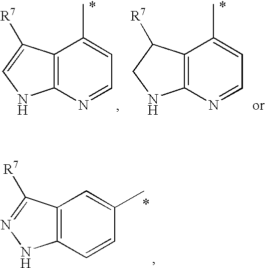
in which,
R⁷ represents hydrogen, halogen, cyano, (C₁-C₆)-alkyl, (C₃-C₆)-cycloalkyl, phenyl or 5- or 6-membered heteroaryl,
where alkyl, cycloalkyl, phenyl or 5- or 6-membered heteroaryl may be substituted by amino, hydroxyl, halogen, (C₁-C₃)-alkyl, (C₁-C₃)-alkoxy or (C₁-C₆)-alkylamino,
and
* represents the point of attachment to Y,
Y represents O or NH,
R¹ and R² independently of one another represent hydrogen, halogen, cyano or (C₁-C₃)-alkyl,
R³ and R⁴ independently of one another represent hydrogen, fluorine, chlorine or methyl,
R⁵ represents hydrogen or (C₁-C₆)-alkyl,
R⁶ represents a radical selected from the group consisting of:
(C₁-C₆)-alkyl which is substituted by amino, hydroxyl, (C₁-C₆)-alkoxy, (C₁-C₆)-alkylthio, (C₁-C₆)-alkylamino, (C₃-C₈)-cycloalkylamino, (C₁-C₆)-alkylcarbonylamino, (C₁-C₆)-alkoxycarbonyl, (C₃-C₈)-cycloalkyl, (C₆-C₁₀)-aryl, 5- to 10-membered heteroaryl or 5- to 10-membered heterocyclyl,
where alkylamino, cycloalkylamino or aryl for their part may be substituted by amino, hydroxyl, halogen, (C₁-C₆)-alkoxy, (C₁-C₆)-alkylamino or (C₆-C₁₀)-aryl,
(C₁-C₆)-alkoxy which may be substituted by amino, hydroxyl or (C₁-C₆)-alkylamino,
dimethylaminoethylamino,
(C₃-C₈)-cycloalkyl, 5- to 10-membered heterocyclyl or 5- to 10-membered heterocyclyloxy,
where cycloalkyl, heterocyclyl or heterocyclyloxy may be substituted by amino, hydroxyl, (C₁-C₆)-alkyl, (C₁-C₆)-alkylamino, oxo or benzyloxy,
and (C₆-C₁₀)-aryl or 5- to 10-membered heteroaryl,
where aryl or heteroaryl may be substituted by amino, hydroxyl, halogen, cyano, (C₁-C₆)-alkyl, which for its part may be substituted by amino or (C₁-C₆)-alkylamino, (C₁-C₆)-alkoxy, (C₁-C₆)-alkylamino or (C₁-C₆)-alkoxycarbonyl,
and its salts, hydrates, hydrates of the salts and solvates.

2. Compound of the formula (I) according to claim 1,
in which
A represents a radical 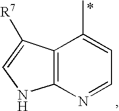
in which
R⁷ represents hydrogen, chlorine or methyl,
and
* represents the point of attachment to Y,
Y represents O,
R¹ and R² independently of one another represent hydrogen, fluorine or chlorine,
R³ and R⁴ independently of one another represent hydrogen or fluorine,
R⁵ represents hydrogen,
R⁶ represents a radical selected from the group consisting of:
(C₁-C₆)-alkyl which is substituted by amino, hydroxyl, (C₁-C₆)-alkoxy, (C₁-C₆)-alkylthio, (C₁-C₆)-alkylamino, (C₅-C₆)-cycloalkylamino, (C₁-C₆)-alkylcarbonylamino, (C₁-C₆)-alkoxycarbonyl, phenyl, 5- or 6-membered heteroaryl or 5- or 6-membered heterocyclyl,
where alkylamino, cycloalkylamino or phenyl for their part may be substituted by hydroxyl, halogen, (C₁-C₃)-alkoxy, (C₁-C₃)-alkylamino or phenyl,
(C₁-C₆)-alkoxy which may be substituted by amino or (C₁-C₆)-alkylamino,
cyclopentyl, cyclohexyl, 5- or 6-membered heterocyclyl or 5- or 6-membered heterocyclyloxy,
where cyclopentyl, cyclohexyl, heterocyclyl or heterocyclyloxy may be substituted by amino, hydroxyl, (C₁-C₃)-alkyl, oxo or benzyloxy,
and phenyl, thienyl, furyl, pyrrolyl, pyrazolyl, thiazolyl, oxazolyl, imidazolyl, pyridyl, pyrimidyl or pyridazinyl,
where phenyl, thienyl, furyl, pyrrolyl, pyrazolyl, thiazolyl, oxazolyl, imidazolyl, pyridyl, pyrimidyl or pyridazinyl may be substituted by amino, hydroxyl, halogen, cyano, (C₁-C₃)-alkyl, which for its part may be substituted by amino or (C₁-C₆)-alkylamino, (C₁-C₃)-alkoxy or (C₁-C₃)-alkoxycarbonyl,
and its salts, hydrates, hydrates of the salts and solvates.

3. Compound of the formula (I) according to claim 1,
in which
A represents a radical 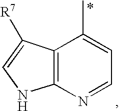
in which
R⁷ represents hydrogen, chlorine or methyl
and
* represents the point of attachment to Y,
Y represents O,
R¹ and R² independently of one another represent hydrogen or fluorine,
R³ and R⁴ represent hydrogen,
R⁵ represents hydrogen,
R⁶ represents a radical selected from the group consisting of:
(C₁-C₆)-alkyl which is substituted by amino, hydroxyl, (C₁-C₆)-alkylamino, cyclohexylamino or piperidinyl,
where alkylamino or cyclohexylamino for their part may be substituted by hydroxyl or phenyl,
(C₁-C₆)-alkoxy which may be substituted by amino or (C₁-C₆)-alkylamino,
cyclopentyl, piperazinyl, piperidinyl, pyrrolidinyl, piperidinyloxy or pyrrolidinyloxy,
where cyclopentyl, piperazinyl, piperidinyl, pyrrolidinyl, piperidinyloxy or pyrrolidinyloxy may be substituted by amino, hydroxyl, (C₁-C₃)-alkyl or benzyloxy,
and phenyl or thienyl,
where phenyl or thienyl may be substituted by (C₁-C₃)-alkyl which for its part may be substituted by amino or (C₁-C₆)-alkylamino,
and its salts, hydrates, hydrates of the salts and solvates.

4. Process for preparing compounds of the formula (I) as defined in claim 1, characterized in that either
[A] compounds of the formula 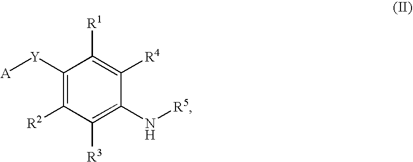
in which
A, Y, R¹, R², R³, R⁴ and R⁵ are as defined in claim 1
are reacted with compounds of the formula 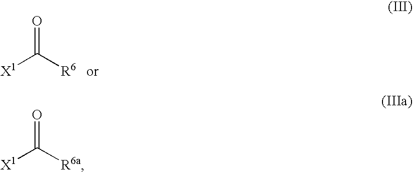
in which
R⁶ is as defined in claim 1,
R^{6a} corresponds to a radical R⁶ as defined above which, however, contains, instead of a secondary or tertiary amino group, a chlorine substituent or, instead of a free amino group, a nitro group or a protected amino group,
and
X¹ represents halogen, preferably chlorine or bromine, or hydroxyl,
and, in the case of the reaction with compounds (Ella) in the radical R^{6a}, the chlorine substituent is subsequently substituted by an amine, the nitro group is hydrogenated to give the corresponding amino group or the protective group is cleaved off to release the corresponding free amino group
or
[B] compounds of the formula 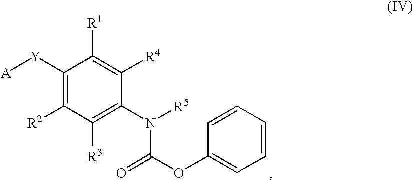
in which
A, Y, R¹, R², R³, R⁴ and R⁵ are as defined in claim 1
are reacted with compounds of the formula
H₂N—R⁸ (V),
in which
R⁸ is as defined in claim 1.

5. Compound as defined in any of claims 1 to 3 for the treatment and/or prophylaxis of disorders.

6. Use of a compound as defined in any of claims 1 to 3 for preparing medicaments for the treatment and/or prophylaxis of cardiovascular disorders.

7. Use of a compound as defined in any of claims 1 to 3 for preparing medicaments for the treatment and/or prophylaxis of erectile dysfunction.

8. Method for the treatment and/or prophylaxis of cardiovascular disorders comprising the use of a cardiovascularly effective amount of a compound as defined in any of claims 1 to 3.

9. Medicament comprising a compound as defined in any of claims 1 to 3 in combination with a further active compound.

10. Medicament comprising a compound as defined in any of claims 1 to 3 in combination with an inert non-toxic pharmaceutically suitable auxiliary.

11. Medicament according to claim 9 or 10 for the treatment and/or prophylaxis of cardiovascular disorders.

12. Medicament according to claim 9 or 10 for the treatment and/or prophylaxis of erectile dysfunction.
